(19) United States
(12) Patent Application Publication
(10) Pub. No.: US 2018/0291036 A1
(43) Pub. Date: Oct. 11, 2018

(54) PROCESS FOR PREPARING THIADIAZOLO-ISOINDOLE-DIONE DERIVATIVES

(71) Applicant: MERCK PATENT GMBH, Darmstadt (DE)

(72) Inventors: Nicolas BLOUIN, Darmstadt (DE); Michal KROMPIEC, Southampton (GB); Andreas LOHR, Dreieich (DE)

(73) Assignee: Merck Patent GmbH, Darmstadt (DE)

(21) Appl. No.: 15/522,515

(22) PCT Filed: Sep. 30, 2015
(86) PCT No.: PCT/EP2015/001927

§ 371 Date: Apr. 27, 2017

(30) Foreign Application Priority Data
Oct. 27, 2014 (EP) 14003638.5

(51) Int. Cl. C07D 513/04; C07D 519/00

(52) U.S. Cl. CPC C07D 513/04; C07D 519/00

(57) ABSTRACT

The invention relates to a novel process for preparing 5H-[1,2,5]thiadiazolo[3,4-f]isoindole-5,7(6H)-dione (“TID”) derivatives, especially for preparing 4,8-diaryl-TID derivatives, to novel intermediates obtained and/or used in this process, to novel TID derivatives prepared by this process, to the use of these TID derivatives as monomers or building blocks for preparing conjugated polymers, and to the use of these TID derivatives or conjugated polymers as organic semiconductors or in organic electronic (OE) devices.

### TECHNICAL FIELD

The invention relates to a novel process for preparing 5H-[1,2,5]thiadiazolo[3,4-f]isoindole-5,7(6M-dione (“TID”) derivatives, especially for preparing 4,8-diaryl-TID derivatives, to novel intermediates obtained and/or used in this process, to novel TID derivatives prepared by this process, to the use of these TID derivatives as monomers or building blocks for preparing conjugated polymers, and to the use of these TID derivatives or conjugated polymers as organic semiconductors or in organic electronic (OE) devices.

### BACKGROUND

In recent years, there has been development of organic semiconducting (OSC) materials in order to produce more versatile, lower cost electronic devices. Such materials find application in a wide range of devices or apparatus, including organic field effect transistors (OFETs), organic light emitting diodes (OLEDs), organic photodetectors (OPDs), organic photovoltaic (OPV) cells, sensors, memory elements and logic circuits to name just a few. The organic semiconducting materials are typically present in the electronic device in the form of a thin layer, for example of between 50 and 300 nm thickness.

One particular area of importance is organic photovoltaics. Conjugated polymers have found use in organic solar cells, for example as electron donor or p-type OSC that is used together with an electron acceptor or n-type OSC, like e.g. a fullerene, in a bulk heterojunction (BHJ) organic solar cell. Conjugated polymers allow OPV devices to be manufactured by solution-processing techniques such as spin casting, dip coating or ink jet printing. Solution processing can be carried out cheaper and on a larger scale compared to the evaporative techniques used to make inorganic thin film devices. Currently, polymer based OPV devices are achieving efficiencies above 8%.

Many high-performance donor polymers for bulk-heterojunction organic solar cells are comprised of alternating donor (electron-rich) and acceptor (electron-poor) blocks, and much research effort is directed towards the development of new donor and acceptor building blocks. One of the promising acceptor building blocks introduced in the recent decade is 5H-[1,2,5]thiadiazolo[3,4-f]isoindole-5,7(6H)-dione TID (1) as shown below, which is usually occurring as a thiophene-flanked unit (2)

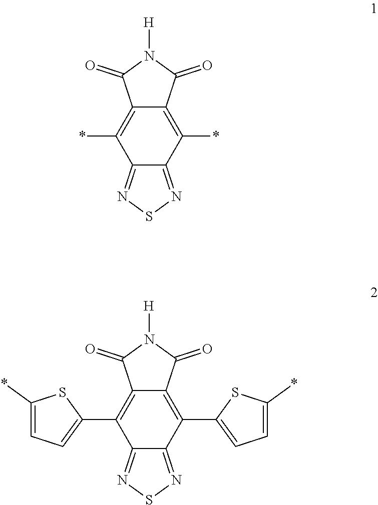 [SMILES: *c1c2c(c(*)c3nsnc13)C(=O)NC2=O ; *c1ccc(-c2c3c(c(-c4ccc(*)s4)c4nsnc24)C(=O)NC3=O)s1]

In prior art several synthesis methods of TID and its derivatives have hitherto been reported, which can be summarized into two synthetic pathways as briefly discussed below.

Pathway A

4,8-Unsubstituted TID was first prepared as a by-product during the synthesis of 2,1,3-benzothiadiazole-5,6-dicarbonitrile by Rosenmund-von Braun cyanation of 5,6-dibromo-2,1,3-benzothiadiazole as shown below

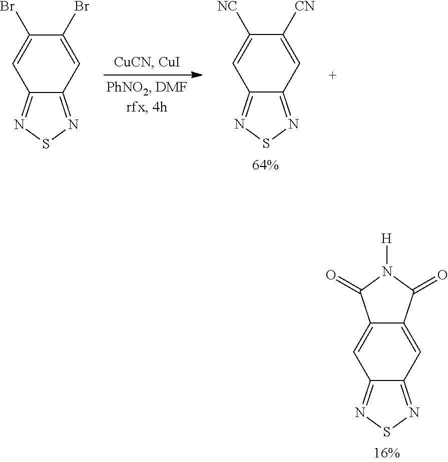 [SMILES: Brc1cc2nsnc2cc1Br ; N#Cc1cc2nsnc2cc1C#N ; O=C1NC(=O)c2cc3nsnc3cc21]

(see E. H. Morkved, S. M. Neset, O. Bjorlo, H. Kjosen, G. Hvistendahl, F. Mo, Acta Chem. Scand., 1995, 49, 658-662 and J. Shao, J. Chang, C. Chi, Org. Biomol. Chem., 2012, 10, 7045).

However, the functionalisation of TID in 4,8 positions is difficult, and direct bromination of TID or N-alkyl-TID has not been reported so far.

N-(2-ethylhexyl)-4,8-dibromo-5H-[1,2,5]thiadiazolo[3,4-f]isoindole-5,7(6H)-dione (N-(2-ethylhexyl-4,8-dibromo-TID) was reported to be obtainable by reductive ring-opening with Fe/AcOH (see J. Shao, J. Chang, C. Chi, Org. Biomol. Chem., 2012, 10, 7045), bromination of the resultant N-(2-ethylhexyl)-5,6-diaminoisoindoline, and subsequent reconstruction of the thiadazole ring by reaction with thionyl chloride, as shown below (see H. Li, T. M. Koh, A. Hagfeldt, M. Graetzel, S. G. Mhaisalkar, A. C. Grimsdale, Chem. Commun., 2013, 2409.

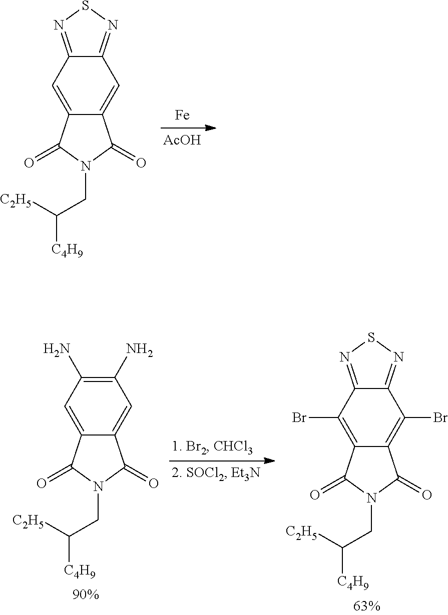

The 4,8-brominated TID can be further functionalised by Stille coupling and subsequent bromination, to yield a thiophene-flanked TID building block as shown below

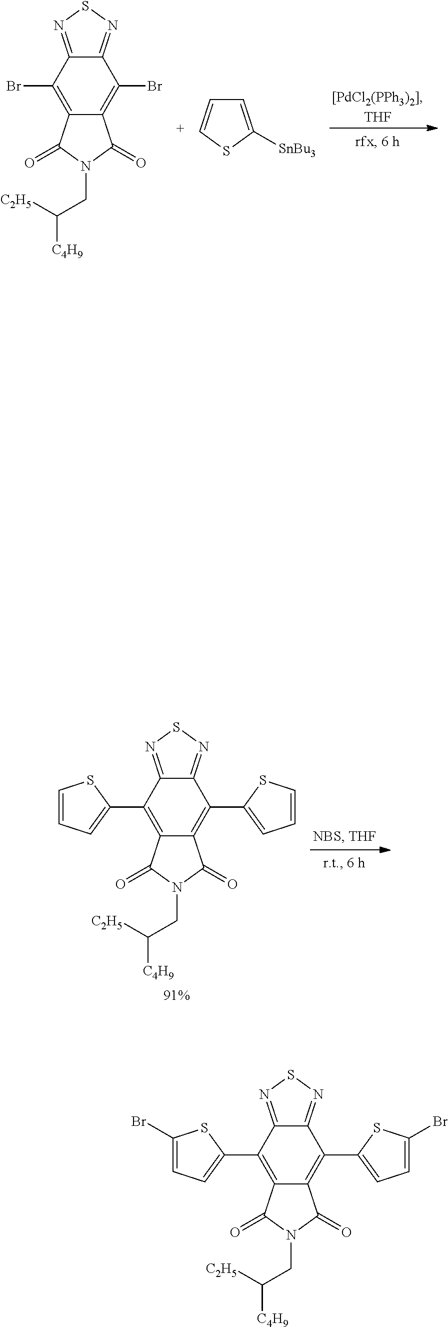

(see H. Li, T. M. Koh, A. Hagfeldt, M. Graetzel, S. G. Mhaisalkar, A. C. Grimsdale, Chem. Commun., 2013, 2409).

However, synthetic pathway A has the disadvantages that it consists of six steps, has a very low yield of TID in the first step (cyanation reaction), and requires the use of toxic organotin compounds (Stille coupling).

Pathway B

WO 2012/149189 discloses an alternative synthetic strategy for N-alkyl-4,8-diaryl-TID that relies on [4+2] cycloaddition of a dimethyl acetylenedicarboxylate to 4,6-bis(5-bromo-2-thienyl)thieno[3,4-c][1,2,5]thiadiazole, conversion of the diester intermediate to an anhydride and, finally, to an imide, as shown below

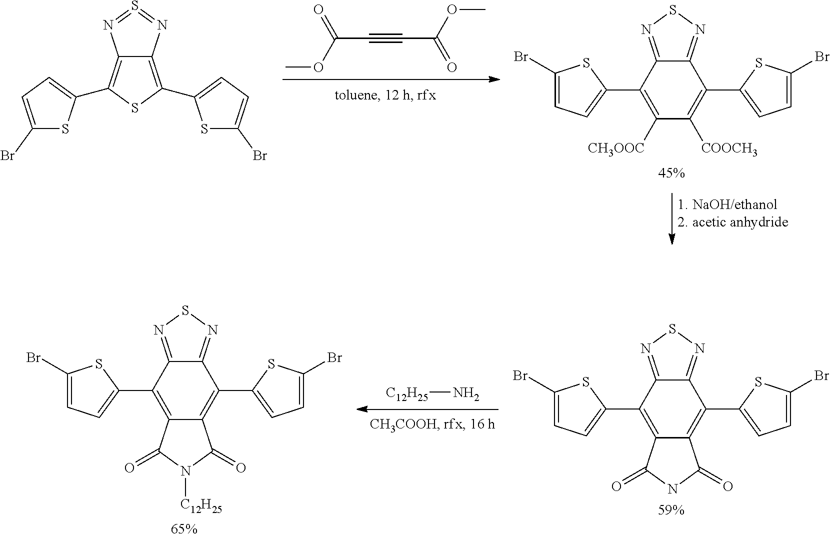

A similar synthesis, starting from 4,6-bis(2-thienyl)thieno[3,4-c][1,2,5]thiadiazole, has been subsequently published by L. Wang, D. Cai, Q. Zheng, C. Tang, S. C. Chen, Z. Yin, ACS Macro Lett., 2013, 2, 605.

WO 2012/149189 also discloses an improved cycloaddition-based method, which consists of a transformation of the thienothiadiazole precursor to the final product in one pot, in a cycloaddition-oxidative cycloreversion sequence as shown below.

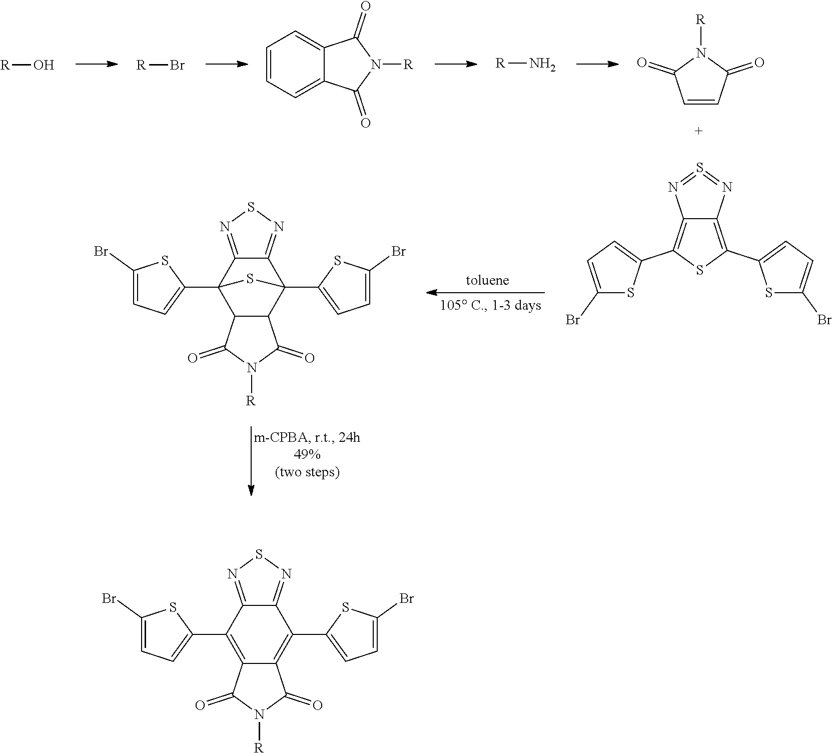

where R is a linear or branched alkyl.

The key intermediate for the two methods mentioned above, 4,6-bis(5-bromo-2-thienyl)thieno[3,4-c][1,2,5]thiadiazole, is prepared in five steps, the key transformations being Stille coupling of 2,5-dibromo-3,4-dinitrothiophene with 2-tributylstannylthiophene, subsequent reduction and annelation of the resultant diaminoterthiophene with PhNSO, as shown below

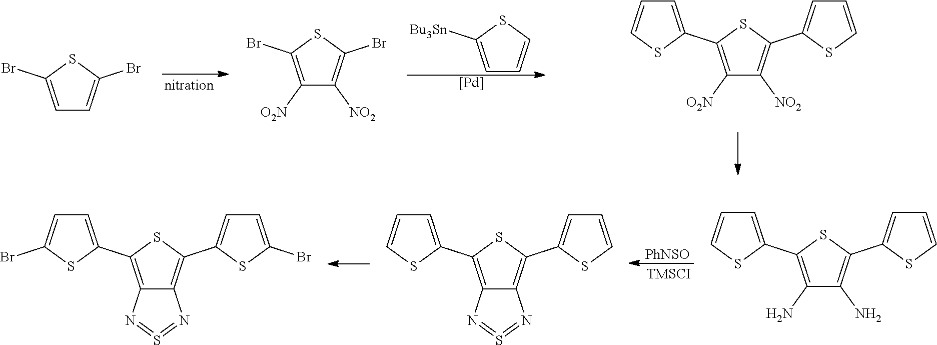

(see J. A. Mikroyannidis, D. V. Tsagkournos, P. Balraju, G. D. Sharma, Sol. En. Mat Sol. Cells, 2011, 95, 3025; and M. C. Ruiz Delgado, V. Hernandez, J. T. Lopez Navarrete, S. Tanaka, Y. Yamashita, J. Phys. Chem. B, 2004, 108, 2516).

However, pathway B has the disadvantages that it consists of nine (in the acetylenedicarboxylate variant) or eleven (in the N-alkylmaleimide variant) steps, when starting from commercially available reagents, and involves the use of highly toxic reagents, like organotin compounds, PhNSO and TMSCl.

For industrial production it is essential to provide a reaction path that allows synthesis at large scale in a time- and cost-effective manner with a low number of individual reaction steps, satisfying yield and purity, without or only with low amounts of undesired side products, and avoids the use of highly toxic or hazardous compounds.

It was therefore an aim of the present invention to provide a process for the synthesis of TID derivatives. especially 4,8-diaryl-TID derivatives, which that does not have the drawbacks of the synthesis methods described in prior art, allows a synthesis with a reduced number of reactions steps, in satisfying yield and purity, without or only with reduced amount of side products, avoids the use of highly toxic or hazardous compounds, and is especially suitable for synthesis at large scale.

The inventors of the present invention have found that these aims could be achieved by providing a process as described and claimed hereinafter.

### SUMMARY

The invention relates to a process of preparing a compound of formula I

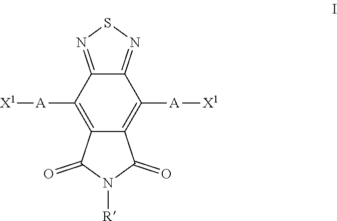

said process comprising the steps of reacting a compound of formula I1

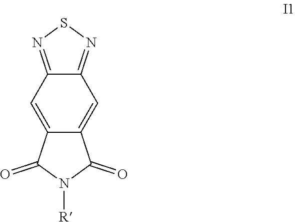

with an aryl- or heteroaryl compound Pg-A-X² to give a compound of formula I2,

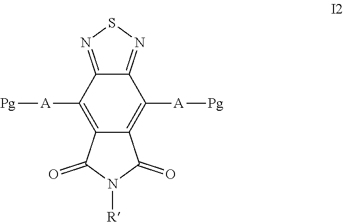

and replacing the groups Pg in the compound of formula I2 by halide groups X¹,
wherein the individual radicals, independently of each other, and on each occurrence identically or differently, have the following meanings
A is arylene or heteroarylene with 5 to 30 ring atoms that is optionally substituted, preferably by one or more groups R^{S},
R′ is H or has one of the meanings of R,
R is straight-chain, branched or cyclic alkyl with 1 to 30 C atoms, in which one or more CH₂ groups are optionally replaced by —O—, —S—, —C(═O)—, —C(═S)—, —C(═O)—O—, —O—C(═O)—, —NR⁰—, —SiR⁰R⁰⁰—, —OCF₂—, —CHR⁰═CR⁰⁰—, —CY¹═CY²— or —C≡C— in such a manner that O and/or S atoms are not linked directly to one another, and in which one or more H atoms are optionally replaced by F, Cl, Br, I or CN, or denotes aryl or heteroaryl with 5 to 15 ring atoms, which is mono- or polycyclic and unsubstituted or substituted by one or more groups R^{S},
R^{S} is F, Br, Cl, —CN, —NC, —NCO, —NCS, —OCN, —SCN, —C(O)NR⁰R⁰⁰, —C(O)X⁰, —C(O)R⁰, —C(O)OR⁰, —NH₂, —NR⁰R⁰⁰, —SH, —SR⁰, —SO₃H, —SO₂R⁰, —OH, —NO₂, —CF₃, —SF₅, optionally substituted silyl, carbyl or hydrocarbyl with 1 to 40 C atoms that is optionally substituted and optionally comprises one or more hetero atoms,
R⁰ and R⁰⁰ are H or optionally substituted C₁₋₄₀ carbyl or hydrocarbyl, and preferably denote H or alkyl with 1 to 12 C-atoms,
X⁰ is halogen, preferably F, Cl or Br,
X¹ is halogen, preferably Br or I,
X² is a leaving group, preferably selected from H, halogen or sulfonate, very preferably Br, I, tosylate, nonaflate or mesylate,
Pg is H or a protecting group, preferably SiMe₃ or Cl.

More specifically, the invention relates to a process of preparing a compound of formula I

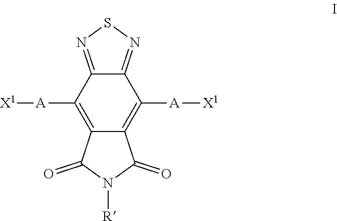

comprising the following steps:
a1) Reacting benzo[2,1,3]thiadiazole 1 that is substituted in 5- and 6-position by Cl, Br, I or sulfonate, preferably by Cl, Br, I, triflate, nonaflate or tosylate, with a cyanating agent, optionally in the presence of a catalyst, to give a product mixture of 5,6-dicyano-benzo[2,1,3]thiadiazole 2 and [1,2,5]thiadiazolo[3,4-e]isoindole-5,7-dione 3, and adding an acid or an acid chloride to the product mixture, and
a2) optionally adding a substituent R to the N-atom in 6-position of the [1,2,5]thiadiazolo[3,4-e]isoindole-5,7-dione 3 to give the N-substituted [1,2,5]thiadiazolo[3,4-e]isoindole-5,7-dione 4,

or, alternatively to steps a1) and a2),
b) reacting benzo[2,1,3]thiadiazole 1 that is substituted in 5- and 6-position by Cl, Br, I or sulfonate, preferably by Cl, Br, I, triflate, nonaflate or tosylate, with a primary amine R—NH₂ and CO in the presence of a catalyst (aminocarbonylation) to give N-substituted [1,2,5]thiadiazolo[3,4-e]isoindole-5,7-dione 4,

and, subsequently to steps a1) and a2) or step b),
c) reacting the [1,2,5]thiadiazolo[3,4-e]isoindole-5,7-dione 3 of step a1), or the N-substituted [1,2,5]thiadiazolo[3,4-e]isoindole-5,7-dione 4 of step a2) or b), with an aryl- or heteroaryl compound Pg-A-X² in the presence of a catalyst and an additive consisting of or comprising a base, to give [1,2,5]thiadiazolo[3,4-e]isoindole-5,7-dione 5 that is 4,8-disubstituted with -A-Pg and optionally N-substituted, and
d) reacting the product 5 from step c) with a halogenating agent containing a halide group X¹, to give 4,8-dihalo-[1,2,5]thiadiazolo[3,4-e]isoindole-5,7-dione 6 that is optionally N-substituted,

wherein the individual radicals are as defined above.

The invention further relates to intermediates obtained by and/or used in a process as described above and below.

The invention further relates to novel compounds of formula I obtainable or obtained by a process as described above and below.

The invention further relates to the use of the compounds of formula I as monomers or building blocks for the preparation of polymers, especially for the preparation of conjugated polymers.

The invention further relates to a conjugated polymer obtained by polymerizing one or more compounds of formula I, optionally together with further co-monomers, preferably in an aryl-aryl coupling reaction.

The invention further relates to the use of a compound of formula I, or a conjugated polymer as described above and below as semiconductor, preferably as electron donor or p-type semiconductor, especially in a semiconducting, charge transport, electrically conducting, photoconducting or light emitting material, or in an optical, electrooptical, electronic, electroluminescent or photoluminescent device, or in a component of such a device or in an assembly comprising such a device or component.

The invention further relates to a semiconducting, charge transport, electrically conducting, photoconducting or light emitting material, which comprises a compound of formula I or a conjugated polymer as described above and below.

The invention further relates to an optical, electrooptical, electronic, electroluminescent or photoluminescent device, or a component thereof, or an assembly comprising it, which comprises a compound of formula I or a conjugated polymer as described above and below, or comprises a semiconducting, charge transport, electrically conducting, photoconducting or light emitting material, as described above and below.

The optical, electrooptical, electronic, electroluminescent and photoluminescent device includes, without limitation, organic field effect transistors (OFET), organic thin film transistors (OTFT), organic light emitting diodes (OLED), organic light emitting transistors (OLET), organic photovoltaic devices (OPV), organic photodetectors (OPD), organic solar cells, dye-sensitized solar cells (DSSC), perovskite-based solar cells, laser diodes, Schottky diodes, photoconductors and photodetectors.

Preferred devices are OFETs, OTFTs, OPVs, OPDs and OLEDs, in particular bulk heterojunction (BHJ) OPVs or inverted BHJ OPVs.

Further preferred is the use of a polymer as described above and below as dye in a DSSC or a perovskite-based solar cell, and a DSSC or perovskite-based solar cells comprising a compound, composition or polymer blend according to the present invention.

The component of the above devices includes, without limitation, charge injection layers, charge transport layers, interlayers, planarising layers, antistatic films, polymer electrolyte membranes (PEM), conducting substrates and conducting patterns.

The assembly comprising such a device or component includes, without limitation, integrated circuits (IC), radio frequency identification (RFID) tags or security markings or security devices containing them, flat panel displays or backlights thereof, electrophotographic devices, electrophotographic recording devices, organic memory devices, sensor devices, biosensors and biochips.

### BRIEF DESCRIPTION OF THE DRAWINGS

FIG. 1 exemplarily and schematically illustrates a preferred process according to the present invention.

### TERMS AND DEFINITIONS

As used herein, the terms “donor” or “donating” and “acceptor” or “accepting” will be understood to mean an electron donor or electron acceptor, respectively. “Electron donor” will be understood to mean a chemical entity that donates electrons to another compound or another group of atoms of a compound. “Electron acceptor” will be understood to mean a chemical entity that accepts electrons transferred to it from another compound or another group of atoms of a compound. See also International Union of Pure and Applied Chemistry, Compendium of Chemical Technology, Gold Book, Version 2.3.2, 19 Aug. 2012, pages 477 and 480.

As used herein, the term “n-type” or “n-type semiconductor” will be understood to mean an extrinsic semiconductor in which the conduction electron density is in excess of the mobile hole density, and the term “p-type” or “p-type semiconductor” will be understood to mean an extrinsic semiconductor in which mobile hole density is in excess of the conduction electron density (see also, J. Thewlis, Concise Dictionary of Physics, Pergamon Press, Oxford, 1973).

As used herein, the term “leaving group” will be understood to mean an atom or group (which may be charged or uncharged) that becomes detached from an atom in what is considered to be the residual or main part of the molecule taking part in a specified reaction (see also Pure Appl. Chem., 1994, 66, 1134).

As used herein, the term “conjugated” will be understood to mean a compound (for example a polymer) that contains mainly C atoms with sp²-hybridisation (or optionally also sp-hybridisation), and wherein these C atoms may also be replaced by hetero atoms. In the simplest case this is for example a compound with alternating C-C single and double (or triple) bonds, but is also inclusive of compounds with aromatic units like for example 1,4-phenylene. The term “mainly” in this connection will be understood to mean that a compound with naturally (spontaneously) occurring defects, or with defects included by design, which may lead to interruption of the conjugation, is still regarded as a conjugated compound.

As used herein, the term “carbyl group” will be understood to mean denotes any monovalent or multivalent organic radical moiety which comprises at least one carbon atom either without any non-carbon atoms (like for example —C≡C—), or optionally combined with at least one non-carbon atom such as N, O, S, P, Si, Se, As, Te or Ge (for example carbonyl etc.). The term “hydrocarbyl group” will be understood to mean a carbyl group that does additionally contain one or more H atoms and optionally contains one or more hetero atoms like for example N, O, S, B, P, Si, Se, As, Te or Ge.

As used herein, the term “hetero atom” will be understood to mean an atom in an organic compound that is not a H- or C-atom, and preferably will be understood to mean N, O, S, B, P, Si, Se, As, Te or Ge.

A carbyl or hydrocarbyl group comprising a chain of three or more C atoms may be straight-chain, branched and/or cyclic, and may include spiro-connected and/or fused rings.

Preferred carbyl and hydrocarbyl groups include alkyl, alkoxy, alkylcarbonyl, alkoxycarbonyl, alkylcarbonyloxy and alkoxycarbonyloxy, each of which is optionally substituted and has 1 to 40, preferably 1 to 25, very preferably 1 to 18 C atoms, furthermore optionally substituted aryl or aryloxy having 6 to 40, preferably 6 to 25 C atoms, furthermore alkylaryloxy, arylcarbonyl, aryloxycarbonyl, arylcarbonyloxy and aryloxycarbonyloxy, each of which is optionally substituted and has 6 to 40, preferably 7 to 40 C atoms, wherein all these groups do optionally contain one or more hetero atoms, preferably selected from N, O, S, B, P, Si, Se, As, Te and Ge.

Further preferred carbyl and hydrocarbyl group include for example: a C₁-C₄₀ alkyl group, a C₁-C₄₀ fluoroalkyl group, a C₁-C₄₀ alkoxy or oxaalkyl group, a C₂-C₄₀ alkenyl group, a C₂-C₄₀ alkynyl group, a C₃-C₄₀ allyl group, a C₄-C₄₀ alkyldienyl group, a C₄-C₄₀ polyenyl group, a C₂-C₄₀ ketone group, a C₂-C₄₀ ester group, a C₆-C₁₈ aryl group, a C₆-C₄₀ alkylaryl group, a C₆-C₄₀ arylalkyl group, a C₄-C₄₀ cycloalkyl group, a C₄-C₄₀ cycloalkenyl group, and the like. Preferred among the foregoing groups are a C₁-C₂₀ alkyl group, a C₁-C₂₀ fluoroalkyl group, a C₂-C₂₀ alkenyl group, a C₂-C₂₀ alkynyl group, a C₃-C₂₀ allyl group, a C₄-C₂₀ alkyldienyl group, a C₂-C₂₀ ketone group, a C₂-C₂₀ ester group, a C₆-C₁₂ aryl group, and a C₄-C₂₀ polyenyl group, respectively.

Also included are combinations of groups having carbon atoms and groups having hetero atoms, like e.g. an alkynyl group, preferably ethynyl, that is substituted with a silyl group, preferably a trialkylsilyl group.

The carbyl or hydrocarbyl group may be an acyclic group or a cyclic group. Where the carbyl or hydrocarbyl group is an acyclic group, it may be straight-chain or branched. Where the carbyl or hydrocarbyl group is a cyclic group, it may be a non-aromatic carbocyclic or heterocyclic group, or an aryl or heteroaryl group.

A non-aromatic carbocyclic group as referred to above and below is saturated or unsaturated and preferably has 4 to 30 ring C atoms. A non-aromatic heterocyclic group as referred to above and below preferably has 4 to 30 ring C atoms, wherein one or more of the C ring atoms are optionally replaced by a hetero atom, preferably selected from N, O, S, Si and Se, or by a —S(O)— or —S(O)₂— group. The non-aromatic carbo- and heterocyclic groups are mono- or polycyclic, may also contain fused rings, preferably contain 1, 2, 3 or 4 fused or unfused rings, and are optionally substituted with one or more groups L, wherein

L is selected from halogen, —CN, —NC, —NCO, —NCS, —OCN, —SCN, —C(═O)NR⁰R⁰⁰, —C(═O)X⁰, —C(═O)R⁰, —NH₂, —NR⁰R⁰⁰, —SH, —SR⁰, —SO₃H, —SO₂R⁰, —OH, —NO₂, —CF₃, —SF₅, optionally substituted silyl, or carbyl or hydrocarbyl with 1 to 40 C atoms that is optionally substituted and optionally comprises one or more hetero atoms, and is preferably alkyl, alkoxy, thioalkyl, alkylcarbonyl, alkoxycarbonyl or alkoxycarbonyloxy with 1 to 20 C atoms that is optionally fluorinated, X⁰ is halogen, preferably F, Cl or Br, and R⁰, R⁰⁰ have the meanings given above and below, and preferably denote H or alkyl with 1 to 12 C atoms.

Preferred substituents L are selected from halogen, most preferably F, or alkyl, alkoxy, oxaalkyl, thioalkyl, fluoroalkyl and fluoroalkoxy with 1 to 16 C atoms, or alkenyl or alkynyl with 2 to 20 C atoms.

Preferred non-aromatic carbocyclic or heterocyclic groups are tetrahydrofuran, indane, pyran, pyrrolidine, piperidine, cyclopentane, cyclohexane, cycloheptane, cyclopentanone, cyclohexanone, dihydro-furan-2-one, tetrahydro-pyran-2-one and oxepan-2-one.

An aryl group as referred to above and below preferably has 4 to 30 ring C atoms, is mono- or polycyclic and may also contain fused rings, preferably contains 1, 2, 3 or 4 fused or unfused rings, and is optionally substituted with one or more groups L as defined above.

A heteroaryl group as referred to above and below preferably has 4 to 30 ring C atoms, wherein one or more of the C ring atoms are replaced by a hetero atom, preferably selected from N, O, S, Si and Se, is mono- or polycyclic and may also contain fused rings, preferably contains 1, 2, 3 or 4 fused or unfused rings, and is optionally substituted with one or more groups L as defined above.

As used herein, “arylene” will be understood to mean a divalent aryl group, and “heteroarylene” will be understood to mean a divalent heteroaryl group, including all preferred meanings of aryl and heteroaryl as given above and below.

Preferred aryl and heteroaryl groups are phenyl in which, in addition, one or more CH groups may be replaced by N, naphthalene, thiophene, selenophene, thienothiophene, dithienothiophene, fluorene and oxazole, all of which can be unsubstituted, mono- or polysubstituted with L as defined above. Very preferred rings are selected from pyrrole, preferably N-pyrrole, furan, pyridine, preferably 2- or 3-pyridine, pyrimidine, pyridazine, pyrazine, triazole, tetrazole, pyrazole, imidazole, isothiazole, thiazole, thiadiazole, isoxazole, oxazole, oxadiazole, thiophene, preferably 2-thiophene, selenophene, preferably 2-selenophene, thieno[3,2-b]thiophene, thieno[2,3-b]thiophene, furo[3,2-b]furan, furo[2,3-b]furan, seleno[3,2-b]selenophene, seleno[2,3-b]selenophene, thieno[3,2-b]selenophene, thieno[3,2-b]furan, indole, isoindole, benzo[b]furan, benzo[b]thiophene, benzo[1,2-b;4,5-bldithiophene, benzo[2,1-b;3,4-b′]dithiophene, quinole, 2-methylquinole, isoquinole, quinoxaline, quinazoline, benzotriazole, benzimidazole, benzothiazole, benzisothiazole, benzisoxazole, benzoxadiazole, benzoxazole, benzothiadiazole, 4H-cyclopenta[2,1-b;3,4-bldithiophene, 7H-3,4-dithia-7-sila-cyclopenta[a]pentalene, all of which can be unsubstituted, mono- or polysubstituted with L as defined above. Further examples of aryl and heteroaryl groups are those selected from the groups shown hereinafter.

An alkyl group or an alkoxy group, i.e., where the terminal CH₂ group is replaced by —O—, can be straight-chain or branched. It is preferably a straight-chain, has 2, 3, 4, 5, 6, 7, 8, 10, 12, 14, 16, 18, 20 or 24 carbon atoms and accordingly is preferably ethyl, propyl, butyl, pentyl, hexyl, heptyl, octyl, decyl, dodecyl, tetradecyl, hexadecyl, octadecyl or didecyl, ethoxy, propoxy, butoxy, pentoxy, hexoxy, heptoxy, octoxy, decoxy, dodecoxy, tetradecoxy, hexadecoxy, octadecoxy or didecoxy, furthermore methyl, nonyl, undecyl, tridecyl, pentadecyl, nonoxy, undecoxy or tridecoxy, for example.

An alkenyl group, wherein one or more CH₂ groups are replaced by —CH═CH— can be straight-chain or branched. It is preferably straight-chain, has 2 to 10 C atoms and accordingly is preferably vinyl, prop-1-, or prop-2-enyl, but-1-, 2- or but-3-enyl, pent-1-, 2-, 3- or pent-4-enyl, hex-1-, 2-, 3-, 4- or hex-5-enyl, hept-1-, 2-, 3-, 4-, 5- or hept-6-enyl, oct-1-, 2-, 3-, 4-, 5-, 6- or oct-7-enyl, non-1-, 2-, 3-, 4-, 5-, 6-, 7- or non-8-enyl, dec-1-, 2-, 3-, 4-, 5-, 6-, 7-, 8- or dec-9-enyl.

Especially preferred alkenyl groups are C₂-C₇-1E-alkenyl, C₄-C₇-3E-alkenyl, C₅-C₇-4-alkenyl, C₆-C₇-5-alkenyl and C₇-6-alkenyl, in particular C₂-C₇-1E-alkenyl, C₄-C₇-3E-alkenyl and C₅-C₇-4-alkenyl. Examples for particularly preferred alkenyl groups are vinyl, 1E-propenyl, 1E-butenyl, 1E-pentenyl, 1E-hexenyl, 1E-heptenyl, 3-butenyl, 3E-pentenyl, 3E-hexenyl, 3E-heptenyl, 4-pentenyl, 4Z-hexenyl, 4E-hexenyl, 4Z-heptenyl, 5-hexenyl, 6-heptenyl and the like. Groups having up to 5 C atoms are generally preferred.

An oxaalkyl group, i.e. where one CH₂ group is replaced by —O—, is preferably straight-chain 2-oxapropyl (=methoxymethyl), 2-(=ethoxymethyl) or 3-oxabutyl (=2-methoxyethyl), 2-, 3-, or 4-oxapentyl, 2-, 3-, 4-, or 5-oxahexyl, 2-, 3-, 4-, 5-, or 6-oxaheptyl, 2-, 3-, 4-, 5-, 6- or 7-oxaoctyl, 2-, 3-, 4-, 5-, 6-, 7- or 8-oxanonyl or 2-, 3-, 4-, 5-, 6-,7-, 8- or 9-oxadecyl, for example. Oxaalkyl, i.e. where one CH₂ group is replaced by —O—, is preferably straight-chain 2-oxapropyl (=methoxymethyl), 2-(=ethoxymethyl) or 3-oxabutyl (=2-methoxyethyl), 2-, 3-, or 4-oxapentyl, 2-, 3-, 4-, or 5-oxahexyl, 2-, 3-, 4-, 5-, or 6-oxaheptyl, 2-, 3-, 4-, 5-, 6- or 7-oxaoctyl, 2-, 3-, 4-, 5-, 6-, 7- or 8-oxanonyl or 2-, 3-, 4-, 5-, 6-,7-, 8- or 9-oxadecyl, for example.

In an alkyl group wherein one CH₂ group is replaced by —O— and one CH₂ group is replaced by —C(O)—, these radicals are preferably neighboured. Accordingly these radicals together form a carbonyloxy group —C(O)—O— or an oxycarbonyl group —O—C(O)—. Preferably this group is straight-chain and has 2 to 6 C atoms. It is accordingly preferably acetyloxy, propionyloxy, butyryloxy, pentanoyloxy, hexanoyloxy, acetyloxymethyl, propionyloxymethyl, butyryloxymethyl, pentanoyloxymethyl, 2-acetyloxyethyl, 2-propionyloxyethyl, 2-butyryloxyethyl, 3-acetyloxypropyl, 3-propionyloxypropyl, 4-acetyloxybutyl, methoxycarbonyl, ethoxycarbonyl, propoxycarbonyl, butoxycarbonyl, pentoxycarbonyl, methoxycarbonylmethyl, ethoxycarbonylmethyl, propoxycarbonylmethyl, butoxycarbonylmethyl, 2-(methoxycarbonyl)ethyl, 2-(ethoxycarbonyl)ethyl, 2-(propoxycarbonyl)ethyl, 3-(methoxycarbonyl)propyl, 3-(ethoxycarbonyl)propyl, 4-(methoxycarbonyI)-butyl.

An alkyl group wherein two or more CH₂ groups are replaced by —O— and/or —C(O)O— can be straight-chain or branched. It is preferably straight-chain and has 3 to 12 C atoms. Accordingly it is preferably bis-carboxy-methyl, 2,2-bis-carboxy-ethyl, 3,3-bis-carboxy-propyl, 4,4-bis-carboxy-butyl, 5,5-bis-carboxy-pentyl, 6,6-bis-carboxy-hexyl, 7,7-bis-carboxy-heptyl, 8,8-bis-carboxy-octyl, 9,9-bis-carboxy-nonyl, 10,10-bis-carboxy-decyl, bis-(methoxycarbonyl)-methyl, 2,2-bis-(methoxycarbonyl)-ethyl, 3,3-bis-(methoxycarbonyl)-propyl, 4,4-bis-(methoxycarbonyl)-butyl, 5,5-bis-(methoxycarbonyl)-pentyl, 6,6-bis-(methoxycarbonyl)-hexyl, 7,7-bis-(methoxycarbonyl)-heptyl, 8,8-bis-(methoxycarbonyl)-octyl, bis-(ethoxycarbonyl)-methyl, 2,2-bis-(ethoxycarbonyl)-ethyl, 3,3-bis-(ethoxycarbonyl)-propyl, 4,4-bis-(ethoxycarbonyl)-butyl, 5,5-bis-(ethoxycarbonyl)-hexyl.

A thioalkyl group, i.e where one CH₂ group is replaced by —S—, is preferably straight-chain thiomethyl (—SCH₃), 1-thioethyl (—SCH₂CH₃), 1-thiopropyl (=—SCH₂CH₂CH₃), 1-(thiobutyl), 1-(thiopentyl), 1-(thiohexyl), 1-(thioheptyl), 1-(thiooctyl), 1-(thiononyl), 1-(thiodecyl), 1-(thioundecyl) or 1-(thiododecyl), wherein preferably the CH₂ group adjacent to the sp² hybridised vinyl carbon atom is replaced.

A fluoroalkyl group is preferably perfluoroalkyl CᵢF₂ᵢ₊₁, wherein i is an integer from 1 to 15, in particular CF₃, C₂F₅, C₃F₇, C₄F₉, C₅F₁₁, C₆F₁₃, C₇F₁₅ or C₈F₁₇, very preferably C₆F₁₃, or partially fluorinated alkyl, in particular 1,1-difluoroalkyl, all of which are straight-chain or branched.

Alkyl, alkoxy, alkenyl, oxaalkyl, thioalkyl, carbonyl and carbonyloxy groups can be achiral or chiral groups. Particularly preferred chiral groups are 2-butyl (=1-methylpropyl), 2-methylbutyl, 2-methylpentyl, 2-ethylhexyl, 2-butylhexyl, 2-ethyloctyl, 2-butyloctly, 2-hexyloctyl, 2-ethyldecyl, 2-butyldecyl, 2-hexyldecyl, 2-octyldecyl, 2-ethyldodecyl, 2-butyldodecyl, 2-hexyldodecyl, 2-octyldodecyl, 2-decyldodecyl, 2-propylpentyl, 3-methylpentyl, 3-ethylpentyl, 3-ethylheptyl, 3-butylheptyl, 3-ethylnonyl, 3-butylnonyl, 3-hexylnonyl, 3-ethylundecyl, 3-butylundecyl, 3-hexylundecyl, 3-octylundecyl, 4-ethylhexyl, 4-ethyloctyl, 4-butyloctyl, 4-ethyldecyl, 4-butyldecyl, 4-hexyldecyl, 4-ethyldodecyl, 4-butyldodecyl, 4-hexyldodecyl, 4-octyldodecyl, in particular 2-methylbutyl, 2-methylbutoxy, 2-methylpentoxy, 3-methyl-pentoxy, 2-ethyl-hexoxy, 2-butyloctoxyo, 2-hexyldecoxy, 2-octyldodecoxy, 1-methylhexoxy, 2-octyloxy, 2-oxa-3-methylbutyl, 3-oxa-4-methyl-pentyl, 4-methylhexyl, 2-hexyl, 2-octyl, 2-nonyl, 2-decyl, 2-dodecyl, 6-methoxy-octoxy, 6-methyloctoxy, 6-methyloctanoyl-oxy, 5-methylheptyloxy-carbonyl, 2-methylbutyryloxy, 3-methylvaleroyloxy, 4-methylhexanoyloxy, 2-chloro-propionyloxy, 2-chloro-3-methylbutyryloxy, 2-chloro-4-methyl-valeryl-oxy, 2-chloro-3-methylvaleryloxy, 2-methyl-3-oxapentyl, 2-methyl-3-oxa-hexyl, 1-methoxypropyl-2-oxy, 1-ethoxypropyl-2-oxy, 1-propoxypropyl-2-oxy, 1-butoxypropyl-2-oxy, 2-fluorooctyloxy, 2-fluorodecyloxy, 1,1,1-trifluoro-2-octyloxy, 1,1,1-trifluoro-2-octyl, 2-fluoromethyloctyloxy for example.

Very preferred are 2-ethylhexyl, 2-butylhexyl, 2-ethyloctyl, 2-butyloctly, 2-hexyloctyl, 2-ethyldecyl, 2-butyldecyl, 2-hexyldecyl, 2-octyldecyl, 2-ethyldodecyl, 2-butyldodecyl, 2-hexyldodecyl, 2-octyldodecyl, 2-decyldodecyl, 3-ethylheptyl, 3-butylheptyl, 3-ethylnonyl, 3-butylnonyl, 3-hexylnonyl, 3-ethylundecyl, 3-butylundecyl, 3-hexylundecyl, 3-octylundecyl, 4-ethyloctyl, 4-butyloctyl, 4-ethyldecyl, 4-butyldecyl, 4-hexyldecyl, 4-ethyldodecyl, 4-butyldodecyl, 4-hexyldodecyl, 4-octyldodecyl, 2-hexyl, 2-octyl, 2-octyloxy, 1,1,1-trifluoro-2-hexyl, 1,1,1-trifluoro-2-octyl and 1,1,1-trifluoro-2-octyloxy.

Preferred achiral branched groups are isopropyl, isobutyl (=methylpropyl), isopentyl (=3-methylbutyl), tert. butyl, isopropoxy, 2-methyl-propoxy and 3-methylbutoxy.

In a preferred embodiment, the alkyl groups are independently of each other selected from primary, secondary or tertiary alkyl or alkoxy with 1 to 30 C atoms, wherein one or more H atoms are optionally replaced by F, or aryl, aryloxy, heteroaryl or heteroaryloxy that is optionally alkylated or alkoxylated and has 4 to 30 ring atoms. Very preferred groups of this type are selected from the group consisting of the following formulae

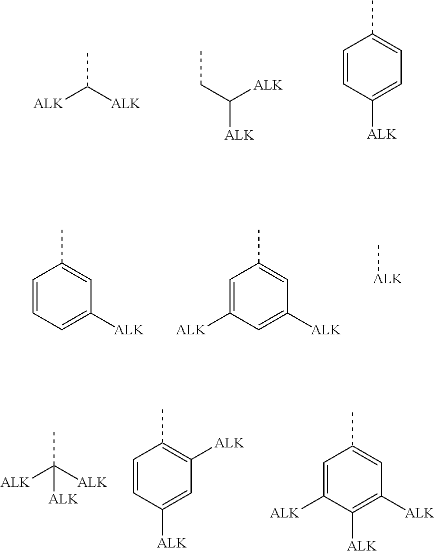

wherein “ALK” denotes optionally fluorinated and straight-chain or branched, preferably straight-chain, alkyl or alkoxy with 1 to 20, preferably 1 to 12 C-atoms, in case of tertiary groups very preferably 1 to 9 C atoms, and the dashed line denotes the link to the ring to which these groups are attached. Especially preferred among these groups are those wherein all ALK subgroups are identical.

—CY¹═CY²— is preferably —CH═CH—, —CF═CF— or —CH═C(CN)—.

X⁰ is halogen, preferably F, Cl or Br.

R⁰ and R⁰⁰ are independently of each other H or optionally substituted C₁₋₄₀ carbyl or hydrocarbyl, and preferably denote H or alkyl with 1 to 12 C-atoms.

If an alkyl or aryl group bis substituted, it is preferably substituted by one or more groups L as defined above.

As used herein, “halogen” includes F, Cl, Br or I. A halogen atom that is used as a substituent that is not intended to take part in a reaction is preferably F or Cl. A halogen atom that is used as a reactive group is preferably Cl, Br or I, most preferably Br or I.

A used herein, —CO—, —C(═O)— and —C(O)— will be understood to mean a carbonyl group, i.e. a group having the structure

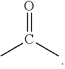

### DETAILED DESCRIPTION

The present invention provides a novel and improved method for the preparation of dihalo-4,8-diaryl-TID compounds, comprising only three or four steps, starting from commercially available compounds. The TID compounds can be used as monomers or building blocks for preparing conjugated polymers.

The process according to the present invention offers significant advantages over prior art, including the following:

the number of synthetic steps, starting from commercially available materials, can be reduced from about 9-11 to 4 steps (via steps a1 and a2) or 3 steps (via step b),

the yield in the step a1) can be considerably improved,

highly toxic organotin reagents can be avoided,

the process via step b) avoids using toxic cyanation reagents such as KCN, NaCN or CuCN.

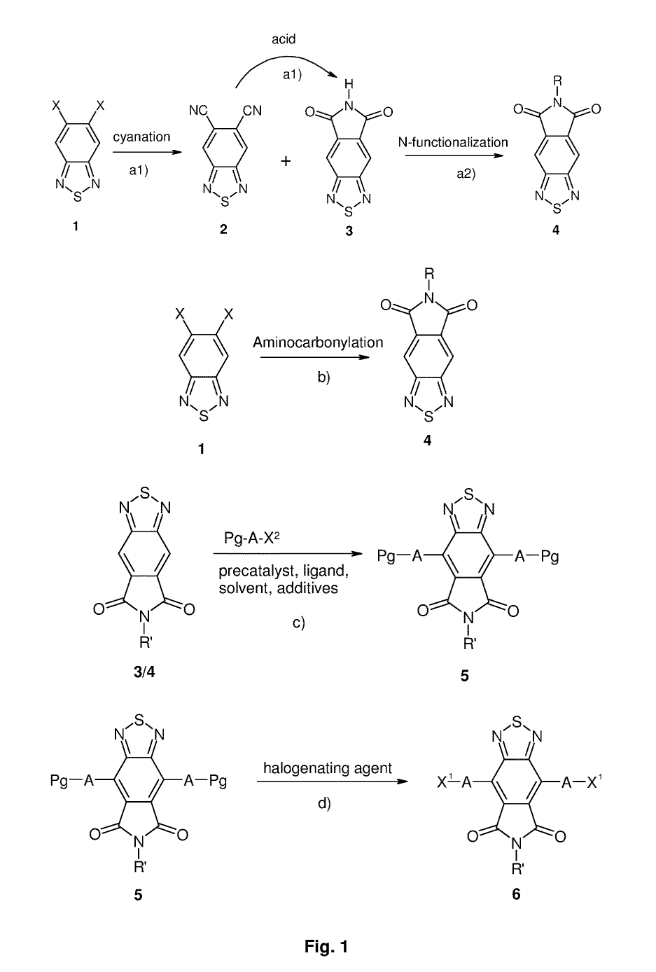

A preferred process according to the present invention, as described in more detail hereinafter, is exemplarily and schematically illustrated in FIG. 1, wherein R, R′, A, Pg, X¹ and X² are as defined in formula I, and X denotes Cl, Br, I or sulfonate, preferably Cl, Br, I, triflate, nonaflate or tosylate, very preferably Br or I.

The first step (step a1) is an improvement over the known procedure for preparation of benzo[2,1,3]-thiadiazole-5,6-dicarbonitrile 2 and TID 3, resulting in increase of the yield of TID, e.g. from 16% up to 51%, and much simpler isolation of the product, since no chromatography is required.

The first step (step a1) comprises cyanation of benzo[2,1,3]thiadiazole 1 that is substituted in 5- and 6-position by Cl, Br, I or sulfonate, preferably by Cl, Br, I, triflate, nonaflate or tosylate, with a cyanating agent to give 5,6-dicyano-benzo[2,1,3]-thiadiazole 2, which is then treated with an acid to give TID 3.

The cyanating agent used in step a1) is preferably a cyanide, very preferably selected from CuCN, KCN, NaCN.

In a preferred embodiment of the present invention, a copper salt like CuI or CuBr is added together with the cyanide to the reaction mixture.

In another preferred embodiment of the present invention, the cyanation in step a1) is carried out in the presence of a catalyst, very preferably a palladium catalyst, which is preferably selected from the catalysts listed below for step c).

Preferably step a1) includes adding an acid or acid chloride in a suitable concentration, preferably 70-100%. The acid is preferably a mineral acid, like for example hydrochloric acid, sulphuric acid, phosphoric acid, nitric acid, or a Lewis acid like for example BF₃. The acid chloride is preferably SOCl₂ or oxalyl chloride.

The acid or acid chloride treatment leads to a significantly improved yield for the conversion of dinitrile 2 to TID 3, compared to the methods as disclosed in prior art, for example from 16% up to 51% as demonstrated in the working examples.

The second step (step a2) is N-functionalization of TID 3 to give N-substituted TID 4, preferably by reacting TID 3 with R—Hal, wherein Hal is halogen, preferably Cl or Br, and R has one of the meanings of formula I or one of the preferred meanings as given above and below.

Preferably the N-functionalization in step a2) is carried out by means of N-alkylation, N-acylation, or N-arylation.

N-alkylation is preferably carried out by reacting TID 3 with an alkyl bromide R—Br in a polar solvent in the presence of base, or in a nonpolar solvent under phase-transfer conditions, or via Mitsunobu reaction, wherein R has one of the meanings of formula I or one of the preferred meanings as given above and below.

N-acylation is preferably carried out by reacting TID 3 with an acid chloride in the presence of DMAP and a base, preferably Et₃N.

N-arylation is preferably carried out by reacting TID 3 in an aromatic nucleophilic substitution, a Buchwald-Hartwig N-arylation, or an Ullmann N-arylation, with R—Br, wherein R has one of the meanings of formula I or one of the preferred meanings as given above and below.

Steps a1) and a2) are preferably carried out in a solvent such as DMF, nitrobenzene, NMP, dimethylacetamide, toluene, xylene (o-, m-, p- or mixtures thereof), mesitylene, isopropylbenzene, dichloromethane.

In another preferred embodiment, instead of a two-step reaction (with steps a1 and a2) the N-substituted TID 4 is prepared directly from benzo[2,1,3]thiadiazole 1 that is substituted in 5- and 6-position by Cl, Br, I or sulfonate, preferably by Cl, Br, I, triflate, nonaflate or tosylate, via transition metal-catalyzed aminocarbonylation reaction (step b), using the desired amine R—NH₂, wherein R has one of the meanings of formula I or one of the preferred meanings as given above and below.

The aminocarbonylation in step b) is preferably carried out in the presence of a palladium catalyst, which is preferably selected from the catalysts listed below for step c).

Step b) is preferably carried out in a solvent such as tetrahydrofuran, 2-methyltetrahydrofuran, DMF, nitrobenzene, NMP, dimethylacetamide, toluene, xylene (o-, m-, p- or mixtures thereof), mesitylene, and isopropylbenzene.

The next and key step of the method according to the present invention (reaction of compound of formula I1 with Pg-A-X², or step c), respectively) comprises a catalyzed direct arylation of TID 3, or N-substituted TID 4, with a protected aryl reagent Pg-A-X², where Pg, A and X² have one of the meanings as given above and below (like for example 2-bromo-5-trimethylsilylthiophene), in the presence of an additive consisting of or comprising a base, to give [1,2,5]thiadiazolo[3,4-e]isoindole-5,7-dione 5 that is 4,8-disubstituted with -A-Pg and optionally N-substituted.

The catalyst used in steps a1), b) and c), or in the reaction of the compound of formula I1 with Pg-A-X², respectively, is added to the reaction mixture in catalytic amounts.

The term “catalytic amount” as used above and below refers to an amount that is clearly below one equivalent of the educt employed, i.e. the compound of formula I1 or the compound 1, 3 or 4, respectively, preferably 0.01 to 10 mol. %, most preferably 0.01 to 2 mol. %, based on the equivalents of the educt employed.

The catalyst used in steps a1), b) and c), or in the reaction of the compound of formula I1 with Pg-A-X², respectively is preferably a metal catalyst, very preferably a palladium(0) catalyst or palladium(II) catalyst.

Preferably the metal catalyst is a palladium(0) catalyst or palladium(II) catalyst that comprises an organic ligand, like for example a trisubstituted phosphine ligand, which is capable of coordinating to the Pd atom.

Preferred phosphine ligands are selected from the formula R^{a}ₓR^{b}_{y} R^{c}_{z}P, wherein P denotes phosphorus, R^{a}, R^{b} and R^{c} are identical or different straight-chain, branched or cyclic alkyl groups with 1 to 12 C atoms that are optionally fluorinated, or aryl groups with 4 to 20 C atoms that are optionally substituted, and x, y and z are 0, 1, 2 or 3, with x+y+z=3.

Examples for suitable and preferred phosphine ligands are triphenylphopshine (PPh₃), tri-tert-butylphosphine (P t-Bu₃), triethylphosphine, tri-iso-propyl-phosphine, tri-cyclohexylphosphine, bis(di-tert-butylphosphino)methane and 2-dicyclohexylphosphino-2′,6′-dimethoxybiphenyl.

Further preferred phosphine ligands are selected of formula Ph₂P(CH₂)ₙPPh₂ where n is an integer from 1 to 5, and any substituted derivatives thereof.

In another preferred embodiment the catalyst is formed from a precatalyst and a ligand, wherein the ligand is capable of coordinating to the Pd atom and is formed in situ in the presence of a base. The precatalyst is preferably a palladium(0) catalyst or palladium(II) catalyst. The ligand is preferably a trisubstituted phosphine ligand, which is capable of coordinating to the Pd atom, and which is formed in situ from a corresponding phosphonium salt by the addition of a base. The base is preferably the base used in the reaction of the compound of formula I1 with Pg-A-X², or in step c).

Preferred phosphonium salts are selected from the formula [R^{a}ₓR^{b}_{y}R^{c}_{z}PH]⁺Z⁻ wherein R^{a-c} and x, y and z are as defined above and Z⁻ is a suitable anion, like for example BF₄⁻, PF₆⁻ or SbF₆⁻. Especially preferred are tetrafluoroborates, like for example P(t-Bu₃)HBF₄, PCy₃HBF₄.

The phosphine ligand or phosphonium salt is added to the reaction mixture preferably in an amount from 0.02 to 10 mol. %, most preferably 0.02 to 2 mol. %, based on the equivalents of the educt employed, i.e. the compound of formula I1 or the compound 1, 3 or 4, respectively. The preferred ratio of Pd:phosphine is 1:2.

Preferred and suitable palladium catalysts are selected from the group consisting of: Palladium(II) pivalate, Acetato(2′-di-t-butylphosphino-1,1′-biphenyl-2-yl)palladium(II), Allylchloro[1,3-bis(2,6-di-i-propylphenyl)-4,5-dihydroimidazol-2-ylidene]palladium(II), Allylchloro[1,3-bis(2,6-di-i-propylphenyl)imidazol-2-ylidene]palladium(II), Allylchloro[1,3-bis(2,4,6-trimethylphenyl)imidazol-2-ylidene]palladium(II), Allylpalladium chloride dimer,(2′-Amino-1,1′-biphenyl-2-yl)methanesulfonatopalladium(II) dimer, Bis[1,2-bis(diphenylphosphino)ethane]palladium(0), Bis(dibenzylidene-acetone)palladium(0), trans-Bis(dicyclohexylamine)bis(acetato)-palladium(II), Bis{[4-(N,N-dimethylamino)phenyl]di-t-butylphosphino}-palladium(0), N,N′-[Bis(2,6-dimethylphenyl)-1,3-dimethyl-1,3-propanediylidene](methyl) (triethylphosphine)palladium(II), [1,3-Bis(2,6-di-i-propylphenyl)-4,5-dihydroimidazol-2-ylidene]{2-[(dimethylamino-kN)methyl]phenyl-kC}(pyridine)palladium(II) tetrafluoroborate, 1,3-Bis(2,6-di-i-propylphenypimidazol-2-ylidene(1,4-naphthoquinone)palladium(0) dimer, [P,P′-1,3-Bis(di-i-propylphosphino)propane][P-1,3-bis(di-i-propylphosphino)propane]palladium(0), Bis(2-methylallyl)palladium chloride dimer, 1,2-Bis(phenylsulfinypethanepalladium(II) acetate, Bis(2,2,6,6-tetramethyl-3,5-heptanedionato)palladium(II), Bis(tri-t-butylphosphine)palladium(0), Bis(tricyclohexylphosphine)palladium(0), [1,3-Bis(2,4,6-trimethylphenyl)-4,5-dihydroimidazol-2-ylidene]{2-[(dimethylamino-kN)methyl]phenyl-kC}(pyridine)palladium (II) tetrafluoroborate, 1,3-Bis(2,4,6-trimethylphenyl)imidazol-2-ylidene(1,4-naphthoquinone)palladium(0) dimer, Bis(tri-o-tolylphosphine)palladium(0), Chloro(2′-amino-1,1′-biphenyl-2-yl)palladium(II) dimer, Chloro(2-di-t-butylphosphino-2′,4′,6′-tri-i-propyl-1,1′-biphenyl)[2-(2-aminoethyl)phenyl]palladium(II), Chloro(2-dicyclohexylphosphino-2′,6′-dimethoxy-1,1′-biphenyl)(2′-amino-1,1′-biphenyl-2-yl) palladium(II), Chloro(2-dicyclohexylphosphino-2′,6′-dimethoxy-1,1′-biphenyl)[2-(2-aminoethylphenyl)]palladium(II) methyl-t-butylether adduct, Chloro(2-dicyclohexylphosphino-3,6-dimethoxy-2′,4′,6′-tri-i-propyl-1,1′-biphenyl)(2′-amino-1,1′-biphenyl-2-yl)palladium(II), Chloro[2-(dicyclohexylphosphino)-3,6-dimethoxy-2′,4′,6′-tri-i-propyl-1,1′-biphenyl][2-(2-aminoethyl)-phenyl]palladium(II), Chloro[2-(dicyclohexylphosphino)-2′-(N,N-dimethylamino)-1,1′-biphenyl](2′-amino-1,1′-biphenyl-2-yl)palladium(II), Chloro(2-dicyclohexylphosphino-2′,6′-di-i-propoxy-1,1′-biphenyl)(2′-amino-1,1′-biphenyl-2-yl)palladium(II),Chloro(2-dicyclohexylphosphino-2′,6′-di-i-propoxy-1,1′-biphenyl)[2-(2-aminoethylphenyl)]palladium(II) methyl-t-butylether adduct, Chloro(2-dicyclohexylphosphino-2′,4′,6′-tri-i-propyl-1,1′-biphenyl)(2′-amino-1,1′-biphenyl-2-yl) palladium(II), Chloro(2-dicyclo-hexylphosphino-2′,4′,6′-tri-i-propyl-1,1′-biphenyl)[2-(2-aminoethyl)phenyl]palladium(II) methyl-t-butylether adduct, Chloro([4-(N,N-dimethyl-amino)phenyl]di-t-butylphosphino}(2′-amino-1,1′-biphenyl-2-yl)palladium(II), Chloro[9,9-dimethyl-4,5-bis(diphenylphosphino)-xanthene][2′-amino-1,1′-biphenyl]palladium(II), Chloro(di-2-norbornylphosphino)(2′-dimethylamino-1,1′-biphenyl-2-yl)palladium(II), Chloro(di-2-norbornylphosphino)(2-dimethylaminomethylferrocen-1-yl)palladium(II), Chloromethyl(1,5-cyclooctadiene)palladium(II), Chloro[(1,2,3-η)-3-phenyl-2-propenyl][1,3-bis(2,6-di-i-propylphenyl)-4,5-dihydroimidazol-2-ylidene]palladium(II), Chloro[(1,2,3-η)-3-phenyl-2-propenyl][1,3-bis(2,6-di-i-propylphenyl)imidazol-2-ylidene]-palladium(II), Cyclopentadienyl[(1,2,3-η)-1-phenyl-2-propenyl]palladium(II), trans-Di(μ-acetato)bis[o-(di-o-tolylphosphino)benzyl]dipalladium(II), Diacetatobis(triphenylphosphine)palladium(II),Diacetato(1,10-phenanthroline)palladium(II), Di-μ-bromobis(tri-t-butylphosphino)di-palladium(I), trans-Dibromobis(triphenylphosphine)-palladium(II),Dibromo(1,5-cyclooctadiene)palladium(II), Dichlorobis(acetonitrile)palladium(II), Dichlorobis(benzo-nitrile)palladium(II), Dichloro[1,1′-bis(di-t-butylphosphino)-ferrocene]palladium(II), Dichloro[1,1′-bis(dicyclohexylphosphino)-ferrocene]palladium(II), Di-μ-chlorobis{2-[(dimethylamino)-methyl]phenyl}dipalladium, Dichlorobis{[4-(N,N-dimethylamino)phenyl]di-t-butylphosphino}palladium(II), Dichloro[2,2′-bis(diphenylphosphino)-1,1′-binaphthyl]palladium(II), Dichloro(1,2-bis(diphenylphosphino)-ethane)palladium(II), Dichloro[1,1′-bis(diphenylphosphino)-ferrocene]palladium(II), Dichloro(1,3-bis(diphenylphosphino)-propane)palladium(II), Dichloro[1,1′-bis(di-i-propylphosphino)ferrocene]-palladium(II), Di-μ-chlorobis[(1,2,3-n)-1-phenyl-2-propenyl]dipalladium(II), trans-Dichlorobis(tricyclohexylphosphine)palladium(II), trans-Dichlorobis(triphenylphosphine)palladium(II), trans-Dichlorobis(tri-o-tolylphosphine)palladium(II), Dichloro(1,5-cyclooctadiene)palladium(II), Dichloro(di-μ-chloro)bis[1,3-bis(2,6-di-i-propylphenyl)imidazol-2-ylidene]dipalladium(II), Dichloro[9,9-dimethyl-4,5-bis(diphenylphosphino)-xanthene]palladium(II), Dichloro(norbornadiene)palladium(II), cis-Dichloro(N,N,N′, N′-tetramethylethylenediamine)palladium(II), cis-Dimethyl(N, N,N′,N′-tetramethylethylenediamine)palladium(II), Methanesulfonato[2-bis(3,5-di(trifluoromethyl)phenylphosphino)-3,6-dimethoxy-2′,4′,6′-tri-i-propyl-1,1′-biphenyl](2′-amino-1,1′-biphenyl-2-yl)palladium(II), Methanesulfonato[di-t-butyl(n-butyl)phosphine](2′-amino-1,1′-biphenyl-2-yl)palladium(II), Methanesulfonato(di-t-butylneopentylphosphine)(2′-amino-1,1′-biphenyl-2-yl)palladium(II), Methanesulfonato(2-(di-t-butylphosphino)-3,6-dimethoxy-2′,4′,6′-tri-i-propyl-1,1′-biphenyl)(2′-amino-1,1′-biphenyl-2-yl)palladium(II), Methanesulfonato(2-(di-t-butylphosphino)-3-methoxy-6-methyl-2′,4′,6′-tri-i-propyl-1,1′-biphenyl)(2′-amino-1,1′-biphenyl-2-yl)palladium(II), Methanesulfonato(2-di-t-butylphosphino-2′,4′,6′-tri-i-propyl-1,1′-biphenyl)(2′-amino-1,1′-biphenyl-2-yl)palladium(II), Methanesulfonato(2-dicyclohexylphosphino-2′,6′-dimethoxy-1,1′-biphenyl)(2′-amino-1,1′-biphenyl-2-yl)palladium(II), Methanesulfonato(2-dicyclohexylphosphino-3,6-dimethoxy-2′,4′,6′-tri-i-propyl-1,1′-biphenyl)(2′-amino-1,1′-biphenyl-2-yl)palladium(II), Methanesulfonato[2-(dicyclohexylphosphino)-2′-(N,N-dimethylamino)-1,1′-biphenyl](2′-amino-1,1′-biphenyl-2-yl)palladium(II), Methanesulfonato(2-dicyclohexylphosphino-2′,6′-di-i-propoxy-1,1′-biphenyl)(2′-amino-1,1′-biphenyl-2-yl)palladium(II), Methanesulfonato{(R)-(−)-1-[(S)-2-(dicyclohexylphosphino)ferrocenyl]ethyldi-t-butylphosphine}(2′-amino-1,1′-biphenyl-2-yl)palladium(II), Methanesulfonato(2-dicyclohexylphosphino-2′,4′,6′-tri-i-propyl-1,1′-biphenyl)(2′-amino-1,1′-biphenyl-2-yl)palladium(II), Methanesulfonato{[4-(N,N-dimethylamino)phenyl]di-t-butylphosphino}(2′-amino-1,1′-biphenyl-2-yl)palladium(II), Methanesulfonato[9,9-dimethyl-4,5-bis(diphenyl-phosphino)xanthene][2′-amino-1,1′-biphenyl]palladium(II), Methanesulfonato(tricyclohexylphosphine)(2′-amino-1,1′-biphenyl-2-yl)palladium(II), (1-Methylallyl)palladium chloride dimer, Palladium(II) acetate, Palladium(II) acetylacetonate, Palladium(II) benzoate, Palladium(II) bromide, Palladium(II) chloride, Palladium(II) trifluoroacetate, Palladium(II) trimethylacetate, Tetrakis(acetonitrile)palladium(II) tetrafluoroborate, Tetrakis(triphenylphosphine)palladium(0),Tris[di(4-acetoxybenzylidene)acetone]dipalladium(0) di(4-acetoxybenzylidene)-acetone adduct, Tris(dibenzylideneacetone)dipalladium(0), Tris{tris[3,5-bis(trifluoromethyl)phenyl]phosphine}palladium(0),

Further preferred palladium catalysts include 0.1-10% palladium on a suitable support, such as activated carbon, charcoal, alumina, barium carbonate, barium sulphate, calcium carbonate, titanium silicate, silica, polyethylenimine/silica), or palladium nanoparticles.

A very preferred catalyst system used in step b) comprises or consists of palladium(II) acetate, palladium(II) chloride or palladium(II) bromide in combination with Xantphos (4,5-Bis(diphenylphosphino)-9,9-dimethylxanthene) or another bidentate phosphine ligand as defined above.

Further suitable and preferred catalysts used in step a1), b) and c), or in the reaction of the compound of formula I1 with Pg-A-X², respectively, are selected from copper(I) and copper(II) salts with Cl, Br or I anions.

A very preferred catalyst system used in step c) comprises or consists of a palladium(II) salt or palladium(II) complex with a ligand such as Cl, Br, acetate or pivalate, in combination with a phosphine ligand or phosphonium salt as defined above.

The base used in step c) can be selected from all aqueous and non-aqueous bases. It is preferable that at least 1.5 equivalents of said base per active hydrogen is present in the reaction mixture. Suitable and preferred bases are, for example, metal alcoholates, or hydroxides, carboxylates, carbonates and phosphates, very preferably carbonates or phosphates, of caesium, an alkali metal or an alkaline earth metal, very preferably a hydroxide, acetate, carbonate, fluoride or phosphate of sodium or potassium. Further preferred are mixtures of one or more of the aforementioned bases. Most preferred is anhydrous Cs₂CO₃, K₂CO₃ or Na₂CO₃.

Very preferably step c) is carried out in the presence of an additive consisting of or comprising a base, which is selected from the following groups

the group consisting of caesium bases, preferably Cs₂CO₃ or CsHCO₃,

the group consisting of anions, which are generated from an acid, preferably pivalic acid (2,2-dimethylpropionic acid), a pivalic acid derivative, or R^{s}—COOH, with R^{S} being as defined above, and an anhydrous base, preferably selected from Na₂CO₃, NaHCO₃, Li₂CO₃, K₂CO₃ or KHCO₃,

the group consisting of additives comprising a silver salt, preferably selected from Ag₂CO₃, Ag₂O, AgNO₃, AgOTf, AgBF₄, AgPF₆, and a base, preferably an anhydrous base or R^{S}₄NOH, with with R^{S} being as defined above, very preferably selected from Na₂CO₃, NaHCO₃, Li₂CO₃, K₂CO₃, KHCO₃.

Suitable and preferred solvents for step c) are selected from DMF, nitrobenzene, NMP, dimethylacetamide, toluene, xylene (o-, m-, p- or mixtures thereof), mesitylene, isopropylbenzene.

The final step (step d) is a deprotection and/or functionalisation, preferably halogenation, of the aryl groups A in 4- and 8-position of TID 5, to yield the 4,8-disubstituted TID 6 of formula I, which is optionally N-substituted, as final product.

If step d) is a deprotection/halogenation, TID 5 is reacted with a bromination or iodination agent, such as NBS, bromine, NIS. In another preferred embodiment, before reaction with the halogenating agent, TID 5 is reacted with a deprotecting agent, such as KF or Bu₄NF.

In a preferred embodiment of the present invention, R′ in the compounds of formula I1, I2, 3/4, 5 and 6 is H.

In another preferred embodiment of the present invention, R′ in the compounds of formula I1, I2, 3/4, 5 and 6 has one of the meanings of R in formula I or one of the preferred meanings of R as given above and below.

Preferably, in the compounds of formula I, I1, I2, 3/4, 5 and 6, R-Hal, R—Br and R—NH₂, R is selected from the group consisting of straight-chain or branched alkyl, alkoxy or sulfanylalkyl with 1 to 30 C atoms, and straight-chain or branched alkylcarbonyl, alkylcarbonyloxy or alkyloxycarbonyl with 2 to 30 C atoms, each of the aforementioned groups being unsubstituted or substituted by one or more F atoms.

Further preferably, in the compounds of formula I, I1, I2, 3/4, 5 and 6, R-Hal, R—Br and R—NH₂, R is selected from the group consisting of aryl, heteroaryl, aryloxy and heteroaryloxy, each of which is optionally fluorinated, alkylated or alkoxylated and has 4 to 30 ring atoms.

In the compounds of formula I X′ is halogen, preferably Br or I.

In the compounds of formula land Pg-A-X², R^{S} preferably denotes, on each occurrence identically or differently, H, straight-chain, branched or cyclic alkyl with 1 to 30 C atoms, in which one or more CH₂ groups are optionally replaced by —O—, —S—, —C(O)—, —C(S)—, —C(O)—O—, —O—C(O)—, —NR⁰—, —SiR⁰ R⁰⁰—, —CF₂—, —CHR⁰=CR⁰⁰—, —CY¹=CY²— or —C≡C— in such a manner that O and/or S atoms are not linked directly to one another, and in which one or more H atoms are optionally replaced by F, Cl, Br, I or CN, or denotes aryl, heteroaryl, aryloxy or heteroaryloxy with 4 to 20 ring atoms which is optionally substituted, preferably by halogen or by one or more of the aforementioned alkyl or cyclic alkyl groups.

In a preferred embodiment, R and R^{S} are selected from primary, secondary or tertiary alkyl or alkoxy with 1 to 30 C atoms, wherein one or more H atoms are optionally replaced by F, or aryl, aryloxy, heteroaryl or heteroaryloxy that is optionally alkylated or alkoxylated and has 4 to 30 ring atoms. Very preferred groups of this type are selected from the group consisting of the following formulae

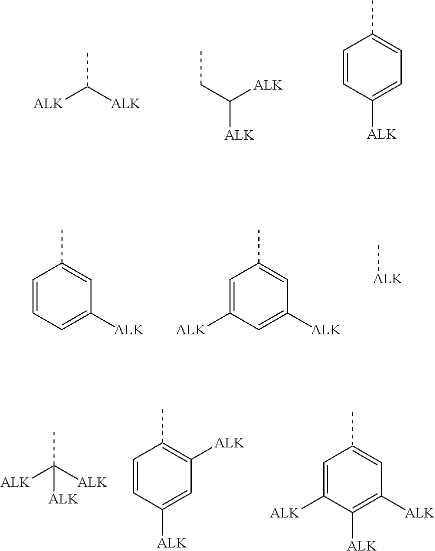

wherein “ALK” denotes optionally fluorinated and straight-chain or branched, preferably straight-chain, alkyl or alkoxy with 1 to 20, preferably 1 to 12 C-atoms, in case of tertiary groups very preferably 1 to 9 C atoms, and the dashed line denotes the link to the ring to which these groups are attached. Especially preferred among these groups are those wherein all ALK subgroups are identical.

In the compounds of formula I and Pg-A-X², A is preferably selected from the following formulae

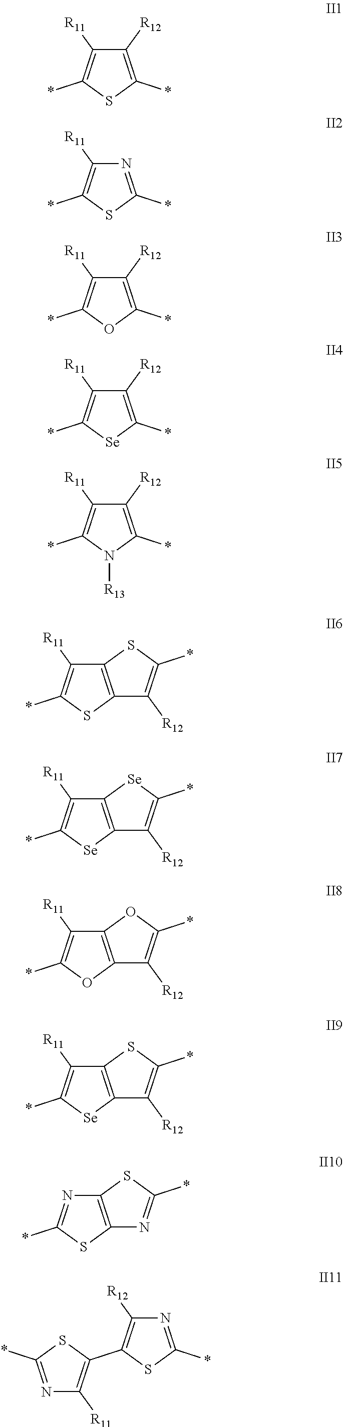

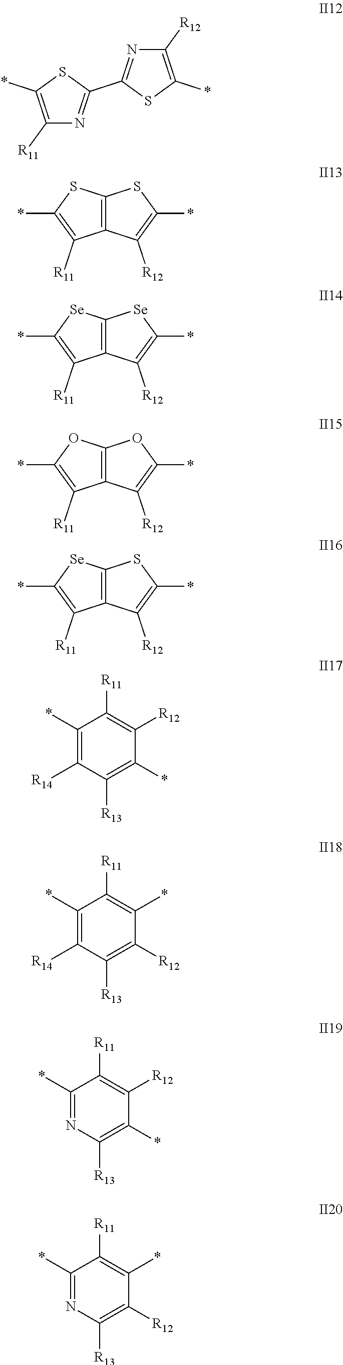

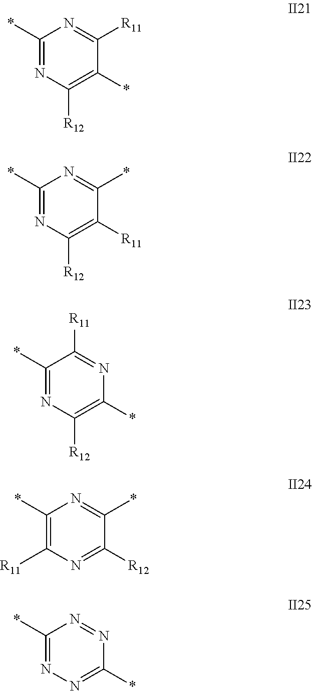

where R¹¹, R¹², R¹³ and R¹⁴ independently of each other denote H or have one of the meanings of R^{S} as defined in formula I or one of the preferred meanings of R^{S} as given above and below.

Preferred are formulae II1 to II10 and II13 to II16. Very preferred are formulae II1 to II10. Most preferred are formulae II1 and II6.

In the compounds of formula Pg-A-X², X² is a leaving group, preferably selected from H, Cl, Br, I, O-tosylate, O-triflate, O-mesylate, O-nonaflate, —O—SO₂Z¹, —Si(Z¹)₃, —SiMe₂F, —SiMeF₂, wherein Me denotes a methyl group, and Z¹ is selected from the group consisting of alkyl, preferably C₁₋₁₀ alkyl and aryl, preferably C₆₋₁₂ aryl, each being optionally substituted, preferably by one or more groups L as defined above, and two groups Z¹ may also form a cyclic group. Especially preferred groups X² are selected from Br, I, O-tosylate, O-triflate, O-mesylate and O-nonaflate.

In the compounds of formula Pg-A-X², Pg is H or a protecting group. If Pg is a protecting group, it is preferably selected from the group consisting of an activated C—H bond, Cl, Br, I, O-tosylate, O-triflate, O-mesylate, O-nonaflate, —O—SO₂Z¹, —Si(Z¹)₃, —SiMe₂F, —SiMeF₂, wherein Me denotes a methyl group, and Z¹ is selected from the group consisting of alkyl, preferably C₁₋₁₀ alkyl and aryl, preferably C₆₋₁₂ aryl, each being optionally substituted, preferably by one or more groups L as defined above, and two groups Z¹ may also form a cyclic group.

Especially preferred groups Pg are Cl, O-tosylate, O-triflate, O-mesylate, O-nonaflate and SiMe₃.

The compounds of formula I are especially suitable as monomers or building blocks for the preparation of polymers, especially for the preparation of conjugated polymers.

The invention thus further relates to a conjugated polymer obtained by polymerizing one or more compounds of formula I, optionally together with further co-monomers.

For example, the conjugated polymer can be suitably prepared by aryl-aryl coupling reactions, such as Yamamoto coupling, C—H activation coupling, Suzuki coupling, Stille coupling, Sonogashira coupling, Heck coupling or Buchwald coupling. Suzuki coupling, Stille coupling and Yamamoto coupling are especially preferred.

Another aspect of the invention is a process for preparing a polymer by coupling one or more identical or different monomers selected from formula I with each other and/or with one or more co-monomers in a polymerisation reaction, preferably in an aryl-aryl coupling reaction.

Preferred aryl-aryl coupling and polymerisation methods used in the processes described above and below are Yamamoto coupling, Kumada coupling, Negishi coupling, Suzuki coupling, Stille coupling, Sonogashira coupling, Heck coupling, C—H activation coupling, Ullmann coupling or Buchwald coupling. Especially preferred are Suzuki coupling, Negishi coupling, Stille coupling and Yamamoto coupling. Suzuki coupling is described for example in WO 00/53656 A1. Negishi coupling is described for example in J. Chem. Soc., Chem. Commun., 1977, 683-684. Yamamoto coupling is described in for example in T. Yamamoto et al., Prog. Polym. Sci., 1993, 17, 1153-1205, or WO 2004/022626 A1. Stille coupling is described for example in Z. Bao et al., J. Am. Chem. Soc., 1995, 117, 12426-12435. C—H activation is described for example for example in M. Leclerc et al, Angew. Chem. Int. Ed. 2012, 51, 2068-2071. For example, when using Yamamoto coupling, monomers having two reactive halide groups are preferably used. When using Suzuki coupling, monomers having two reactive boronic acid or boronic acid ester groups or two reactive halide groups are preferably used. When using Stille coupling, monomers having two reactive stannane groups or two reactive halide groups are preferably used. When using Negishi coupling, monomers having two reactive organozinc groups or two reactive halide groups are preferably used. When synthesizing a linear polymer by C—H activation polymerisation, preferably a monomer as described above is used wherein at least one reactive group is a activated hydrogen bond.

Preferred catalysts, especially for Suzuki, Negishi or Stille coupling, are selected from Pd(0) complexes or Pd(II) salts. Preferred Pd(0) complexes are those bearing at least one phosphine ligand such as Pd(Ph₃P)₄. Another preferred phosphine ligand is tris(ortho-tolyl)phosphine, i.e. Pd(o-Tol₃P)₄. Preferred Pd(II) salts include palladium acetate, i.e. Pd(OAc)₂ or trans-di(μ-acetato)-bis[o-(di-o-tolylphosphino)benzyl]dipalladium(II). Alternatively the Pd(0) complex can be prepared by mixing a Pd(0) dibenzylideneacetone complex, for example tris(dibenzyl-ideneacetone)dipalladium(0), bis(dibenzylideneacetone)palladium(0), or Pd(II) salts e.g. palladium acetate, with a phosphine ligand, for example triphenylphosphine, tris(ortho-tolyl)phosphine, tris(o-methoxyphenyl)phosphine or tri(tert-butyl)phosphine. Suzuki polymerisation is performed in the presence of a base, for example sodium carbonate, potassium carbonate, cesium carbonate, lithium hydroxide, potassium phosphate or an organic base such as tetraethylammonium carbonate or tetraethylammonium hydroxide. Yamamoto polymerisation employs a Ni(0) complex, for example bis(1,5-cyclooctadienyl) nickel(0).

Suzuki, Stille or C—H activation coupling polymerisation may be used to prepare homopolymers as well as statistical, alternating and block random copolymers. Statistical, random block copolymers or block copolymers can be prepared for example from the above monomers, wherein one of the reactive groups is halogen and the other reactive group is a C—H activated bond, boronic acid, boronic acid derivative group or and alkylstannane. The synthesis of statistical, alternating and block copolymers is described in detail for example in WO 03/048225 A2 or WO 2005/014688 A2.

Unless the context clearly indicates otherwise, as used herein plural forms of the terms herein are to be construed as including the singular form and vice versa.

Throughout the description and claims of this specification, the words “comprise” and “contain” and variations of the words, for example “comprising” and “comprises”, mean “including but not limited to”, and are not intended to (and do not) exclude other components.

It will be appreciated that variations to the foregoing embodiments of the invention can be made while still falling within the scope of the invention. Each feature disclosed in this specification, unless stated otherwise, may be replaced by alternative features serving the same, equivalent or similar purpose. Thus, unless stated otherwise, each feature disclosed is one example only of a generic series of equivalent or similar features.

All of the features disclosed in this specification may be combined in any combination, except combinations where at least some of such features and/or steps are mutually exclusive. In particular, the preferred features of the invention are applicable to all aspects of the invention and may be used in any combination. Likewise, features described in non-essential combinations may be used separately (not in combination).

Above and below, unless stated otherwise percentages are percent by weight and temperatures are given in degrees Celsius.

The invention will now be described in more detail by reference to the following examples, which are illustrative only and do not limit the scope of the invention.

### EXAMPLE 1

### 4,8-Bis-(5-bromo-thiophen-2-yl)-6-(2-octyl-dodecyl)-[1,2,5]thiadiazolo[3,4-e]isoindole-5,7-dione is prepared as follows.

### Step a1): [1,2,5]Thiadiazolo[3,4-e]isoindole-5,7-dione

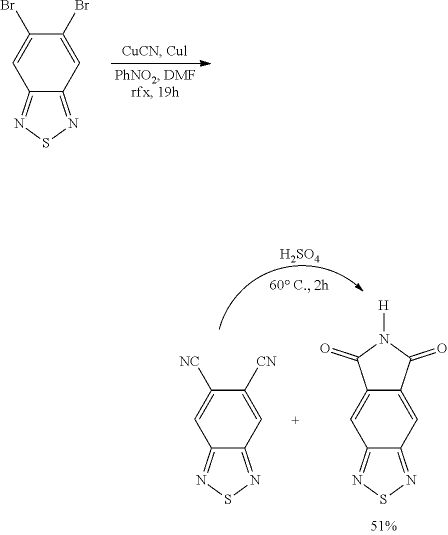 [SMILES: Brc1cc2nsnc2cc1Br ; "H2SO4" ; N#Cc1cc2nsnc2cc1C#N ; O=C1NC(=O)c2cc3nsnc3cc21]

A mixture of nitrobenzene (400 cm³) and dry N,N-dimethylformamide (1250 cm³) is added to 5,6-dibromo-benzo[2,1,3]thiadiazole (21.11 g; 71.81 mmol; 1.00 eq.), copper cyanide (26.37 g; 294.42 mmol; 4.10 eq.) and copper iodide (14.36 g; 75.40 mmol; 1.05 eq.). The mixture is stirred under reflux for 19 h. Subsequently, the mixture is cooled to 23° C. and a mixture of hydrated iron(III) chloride hexahydrate (71.82 g; 265.70 mmol; 3.70 eq.), concentrated (34-36%) hydrochloric acid (18 cm³) and water (106 cm³) is slowly added. The resultant suspension is heated at 70° C. for 1 h, cooled, diluted with water (500 cm³) and dichloromethane (400 cm³), and filtered. The filtrate is separated, the aqueous phase is extracted with dichloromethane (3×500 cm³). Combined organic phases are treated with solid sodium hydrogen carbonate and, subsequently, dried with magnesium sulphate and filtered. The solvents are removed in vacuo (95° C., ca. 6 mbar). The resultant solid is washed with methanol (500 cm³), dried in air and suspended in 96% sulphuric acid (360 cm³). The mixture is heated to 60° C. for 90 minutes and subsequently poured into ice. The solid is filtered, washed with water (1000 cm³) and methanol (200 cm³) and dried in vacuo, yielding a light-grey powder (6.82 g, 51%) ¹H-NMR (300 MHz, DMSO, δ ppm): 11.86 (s, 1H), 8.59 (s, 2H).

### Step a2): 6-(2-Octyl-dodecyl)-[1,2,5]thiadiazolo[3,4-e]isoindole-5,7-dione

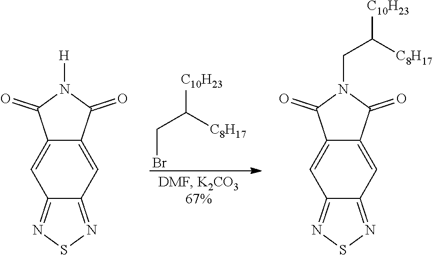

[1,2,5]Thiadiazolo[3,4-e]isoindole-5,7-dione (2.00 g; 9.75 mmol), potassium carbonate (4.04 g; 29.24 mmol; 3.00 eq.) and 9-bromomethyl-nonadecane (4.93 g; 13.65 mmol; 1.40 eq.) are heated in N,N-dimethylformamide (61 cm³) at 140° C. for 20 h, under nitrogen. The reaction is cooled to room temperature and the solvent removed in vacuo. The residue is taken up in dichloromethane (50 cm³), and washed with 10% aqueous hydrochloric acid (1×100 cm³). The aqueous phase is extracted with dichloromethane (2×50 cm³). Combined organic phases are treated with solid sodium hydrogen carbonate and, after foaming finished, dried over magnesium sulphate and filtered. The solvent is removed in vacuo and the residue purified by silica gel column chromatography using petroleum ether (b.p. 40-60° C.) and dichloromethane as eluents. Yield: 3.34 g (67.1%).

¹H-NMR (300 MHz, CDCl₃, δ ppm): 8.502 (s, 2H), 3.68 (d, 2H, J=7.3 Hz), 1.94 (m, 1H), 1.28 (m, 30H), 0.86 (m, 6H).

### Step c): 6-(2-Octyl-dodecyl)-4,8-bis-(5-trimethylsilanyl-thiophen-2-yl)-[1,2,5]thiadiazolo[3,4-e]isoindole-5,7-dione

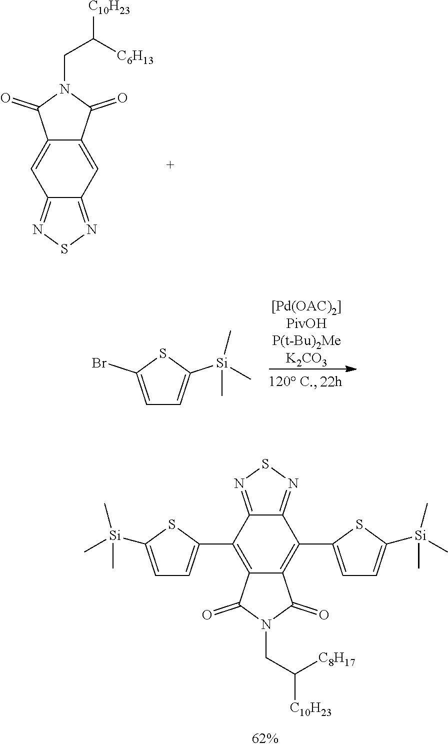

A glass vial is charged with 6-(2-octyl-dodecyl)-[1,2,5]thiadiazolo[3,4-e]isoindole-5,7-dione (500 mg; 1.03 mmol), (5-bromothiophen-2-yl)-trimethyl-silane (651 mg; 2.77 mmol; 2.7 eq.), 2,2-dimethylpropionic acid (105 mg; 1.03 mmol; 1.0 eq.), palladium(II) acetate (23 mg; 0.10 mmol; 0.1 eq.), di-tert-butyl-methyl-phosphane tetrafluoroborate (51 mg; 0.21 mmol; 0.2 eq.), potassium carbonate (426 mg; 3.1 mmol; 3.0 eq.). The vial is sealed and degassed. Toluene (1.20 cm³) is added and the vial heated to 120° C. for 22 h, under nitrogen. The mixture is cooled to room temperature, diluted with dichloromethane (50 cm³), filtered and the solvent removed in vacuo. The residue is dissolved in cyclohexane (15 cm³) and purified by column chromatography on silica, using petroleum ether (b.p. 40-60° C.) and dichloromethane as eluents. Yield: 508 mg, orange oil (62%).

¹H-NMR (300 MHz, CDCl₃, δ ppm): 7.93 (d, 2H, J=3.6 Hz), 7.39 (d, 2H, J=3.6 Hz), 3.62 (d, 2H, J=7.4 Hz), 1.94 (m, 1H), 1.26 (m, 30 H), 0.86 (m, 6H), 0.41 (m, 18H).

### Step d): 4,8-Bis-(5-bromo-thiophen-2-yl)-6-(2-octyl-dodecyl)-[1,2,5]thiadiazolo[3,4-e]isoindole-5,7-dione

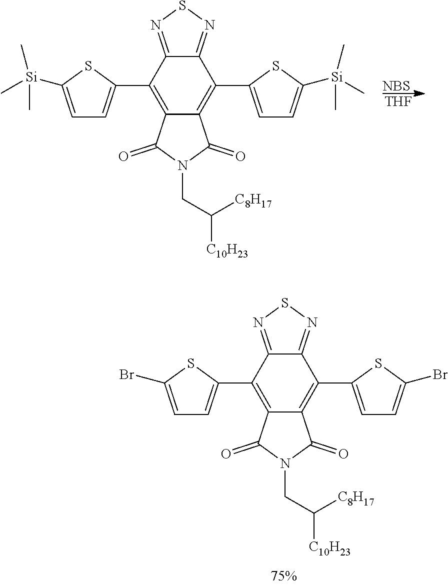

6-(2-Octyl-dodecyl)-4,8-bis-(5-trimethylsilanyl-thiophen-2-yl)-[1,2,5]thiadiazolo[3,4-e]isoindole-5,7-dione (508 mg; 0.64 mmol) is dissolved in tetrahydrofuran (20 cm³). 1-Bromo-pyrrolidine-2,5-dione (233 mg; 1.31 mmol; 2.05 eq.) is added in one portion and the mixture stirred for 18 h. The solvent is removed on a rotary evaporator and the residue is dissolved in dichloromethane (100 cm³), washed with water (100 cm³) and dried over MgSO₄. The solution is filtered and the solvent removed in vacuo. The residue is purified by column chromatography on silica, using petroleum ether (40-60° C.) and dichloromethane as eluents. Yield 389 mg (75%).

¹H-NMR (300 MHz, CDCl₃, δ ppm): 7.78 (d, 2H, J=4.1 Hz), 7.22 (d, 2H, J=4.1 Hz), 3.63 (d, 2H, J=7.3 Hz), 1.92 (m, 1H), 1.25 (m, 30H), 0.86 (m, 6H).

### EXAMPLE 2

### 4,8-Bis-(5-bromo-thiophen-2-yl)-6-(2-ethyl-hexyl)-[1,2,5]thiadiazolo[3,4-e]isoindole-5,7-dione is prepared as follows.

### Step b): 6-(2-Ethyl-hexyl)-[1,2,5]thiadiazolo[3,4-e]isoindole-5,7-dione

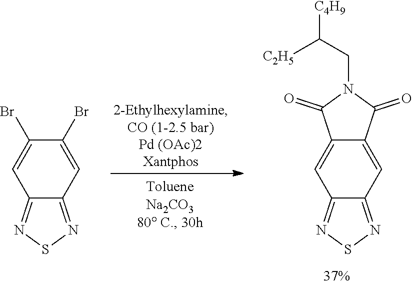

A glass autoclave is charged with 5,6-dibromo-benzo[2,1,3]thiadiazole (2.00 g; 6.67 mmol; 1.00 eq.), 2-ethylhexylamine (0.862 g; 6.67 mmol; 1.0 eq.), palladium(II)acetate (30.0 mg), Xantphos (80.0 mg), Na₂CO₃ (1.60 g; 15.1 mmol; 2.3 eq.) and toluene (17.4 g). A CO pressure of 1 to 2.3 bar is applied and the reaction mixture is stirred at 80° C. for 30 h. The reaction mixture is filtered over silica and the filtrate is concentrated in vacuo. Yield: 780 mg, yellow oil that crystallizes on standing (37%).

¹H-NMR (300 MHz, CDCl₃, δ ppm): 8.50 (s, 1H), 3.70 (d, J=7.4 Hz, 2H), 2.00-1.81 (m, 1H), 1.45-1.19 (m, 8H), 0.93 (m, 6H).

### Step c): 6-(2-Ethyl-hexyl)-4,8-bis-(5-trimethylsilanyl-thiophen-2-yl)-[1,2,5]thiadiazolo[3,4-e]isoindole-5,7-dione

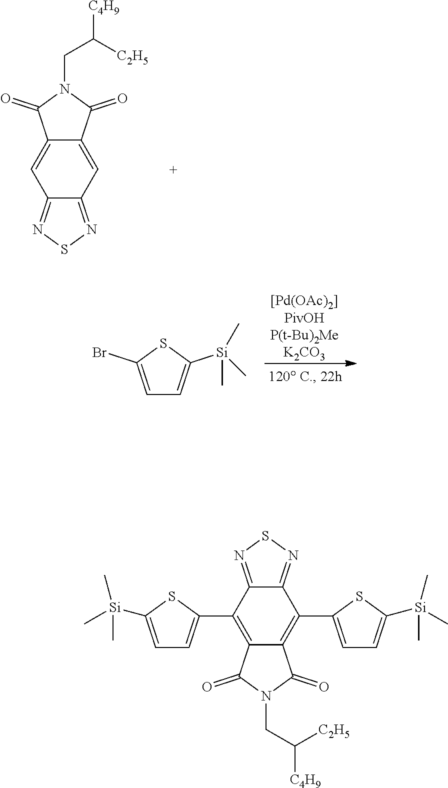

The reaction is carried out in analogy to step c) of Example 1. A glass vial is charged with 6-(2-Ethyl-hexyl)-[1,2,5]thiadiazolo[3,4-e]isoindole-5,7-dione (500 mg; 1.51 mmol), (5-bromothiophen-2-yl)-trimethyl-silane (954 mg; 4.06 mmol; 2.7 eq.), 2,2-dimethylpropionic acid (154 mg; 1.51 mmol; 1.0 eq.), palladium(II) acetate (33 mg; 0.15 mmol; 0.1 eq.), di-tert-butyl-methyl-phosphane tetrafluoroborate (74 mg; 0.30 mmol; 0.2 eq.), potassium carbonate (625 mg; 4.53 mmol; 3.0 eq.). The vial is sealed and degassed. Toluene (1.77 cm³) is added and the vial heated to 120° C. for 22h, under nitrogen. The mixture is cooled to room temperature, diluted with dichloromethane (50 cm³), filtered and the solvent removed in vacuo. The residue is dissolved in cyclohexane (15 cm³) and purified by column chromatography on silica, using petroleum ether (b.p. 40-60° C.) and dichloromethane as eluents.

### Step d): 4,8-Bis-(5-bromo-thiophen-2-yl)-6-(2-ethyl-hexyl)-[1,2,5]thiadiazolo[3,4-e]isoindole-5,7-dione (theoretical example)

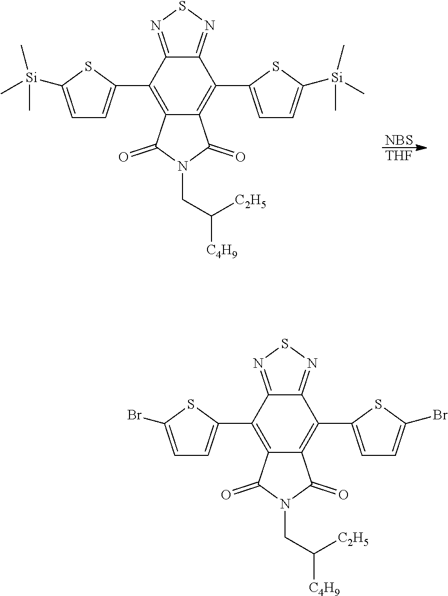

The reaction is carried out in analogy to step d) of Example 1. 6-(2-Ethyl-hexyl)-4,8-bis-(5-trimethylsilanyl-thiophen-2-yl)-[1,2,5]thiadiazolo[3,4-e]isoindole-5,7-dione (401 mg; 0.64 mmol) is dissolved in tetrahydrofuran (20 cm³). 1-Bromo-pyrrolidine-2,5-dione (234 mg; 1.31 mmol; 2.05 eq.) is added in one portion and the mixture stirred for 18 h. The solvent is removed on a rotary evaporator and the residue is dissolved in dichloromethane (100 cm³), washed with water (100 cm³) and dried over MgSO₄. The solution is filtered and the solvent removed in vacuo. The residue is purified by column chromatography on silica, using petroleum ether (40-60° C.) and dichloromethane as eluents.

### EXAMPLE 3

### 4,8-Bis-(5-bromo-thieno[3,2-b]thiophen-2-yl)-6-(3,7-dimethyl-octyl)-[1,2,5]thiadiazolo[3,4-e]isoindole-5,7-dione is prepared as follows:

### Step a1): [1,2,5]Thiadiazolo[3,4-e]isoindole-5,7-dione is prepared as in step a1) of Example 1.

### Step a2): 6-(3,7-Dimethyl-octyl)-[1,2,5]thiadiazolo[3,4-e]isoindole-5,7-dione

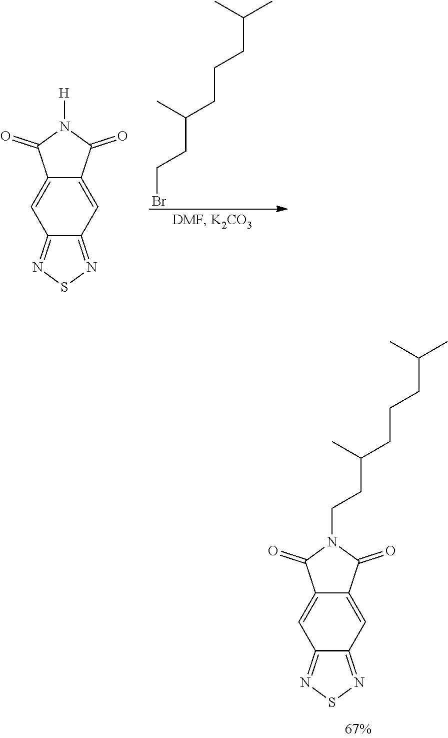

[1,2,5]Thiadiazolo[3,4-e]isoindole-5,7-dione (4.00 g; 19.49 mmol), potassium carbonate (8.08 g; 58.48 mmol; 3.00 eq.) and 1-bromo-3,7-dimethyl-octane (6.04 g; 5.66 cm³; 27.29 mmol; 1.40 eq.) are heated in dimethylformamide (122 cm³) at 140° C. for 20 h, under nitrogen. The reaction is cooled to room temperature and the solvent removed in vacuo. The residue is taken up in dichloromethane (50 cm³), and washed with 10% aqueous hydrochloric acid (1×100 cm³). The aqueous phase is extracted with dichloromethane (2×50 cm³). Combined organic phases are treated with solid sodium hydrogen carbonate and, after foaming finished, dried over magnesium sulphate and filtered. The solvent is removed in vacuo and the residue purified by silica gel column chromatography using petroleum ether (b.p. 40-60° C.) and dichloromethane as eluents. Yield: 4.45 g (66.1%).

¹H-NMR (300 MHz, CDCl₃, δ ppm): 8.50 (s, 2H), 3.81 (m, 2H), 1.75 (m, 1H), 1.55 (m, 4H), 1.09-1.41 (m, 9H), 0.99 (d, 3H, J=6.3 Hz), 0.86 (d, 6H, J=6.6 Hz).

### Step c): 6-(3,7-Dimethyl-octyl)-4,8-bis-(5-trimethylsilanyl-thieno[3,2-b]thiophen-2-yl)-[1,2,5]thiadiazolo[3,4-e]isoindole-5,7-dione

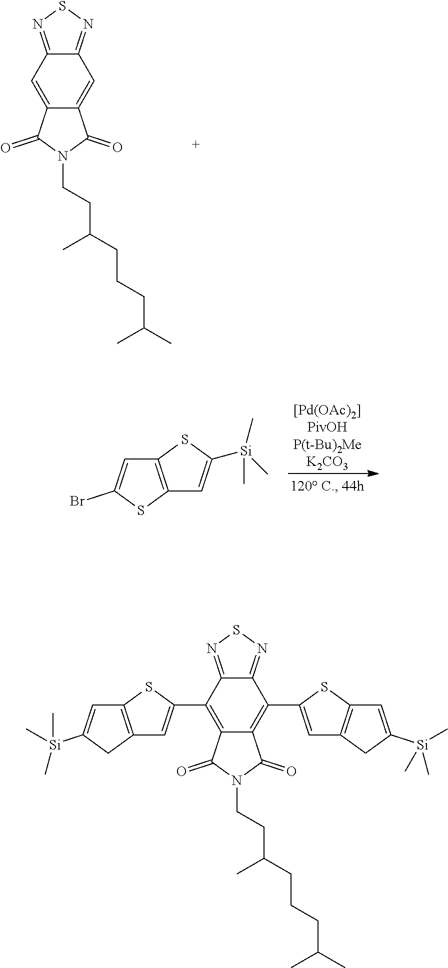

A flask is charged with 6-(3,7-dimethyl-octyl)-[1,2,5]thiadiazolo[3,4-e]isoindole-5,7-dione (737 mg; 2.13 mmol), (5-bromo-thieno[3,2-b]thiophen-2-yl)-trimethyl-silane (1540 mg; 5.39 mmol; 2.48 eq.), 2,2-dimethylpropionic acid (218 mg; 2.13 mmol; 1.0 eq.), palladium(II) acetate (96 mg; 0.43 mmol; 0.2 eq.), di-tert-butyl-methyl-phosphane tetrafluoroborate (212 mg; 0.85 mmol; 0.4 eq.), potassium carbonate (884 mg; 6.4 mmol; 3.0 eq.) and degassed. Toluene (2.50 cm³) is added and the flask heated to 120° C. for 22 h, under nitrogen. The mixture is cooled to room temperature and another portion of (5-bromo-thieno[3,2-b]thiophen-2-yl)-trimethyl-silane (621 mg; 2.13 mmol; 1.0 eq.), palladium(II) acetate (96 mg; 0.43 mmol; 0.2 eq.), di-tert-butyl-methyl-phosphane tetrafluoroborate (212 mg; 0.85 mmol; 0.4 eq.) and potassium carbonate (118 mg; 0.85 mmol; 0.4 eq.) is added and the flask heated under nitrogen for 22 h. The mixture is subsequently cooled to room temperature, diluted with dichloromethane (200 cm³), filtered and the solvent removed in vacuo. The residue is dissolved in cyclohexane/dichloromethane (9:1 v/v) and purified by column chromatography on silica, using petroleum ether (b.p. 40-60° C.) and dichloromethane as eluents. Yield: 784 mg, red oil (48%).

¹H-NMR (300 MHz, CDCl₃, δ ppm): 8.05 (s, 2H), 7.45 (s, 2H), 3.76 (m, 2H), 1.69 (m, 1H), 1.48 (m, 4H), 1.05-1.35 (m, 6H), 0.95 (d, 3H, J=6.1 Hz), 0.84 (d, 6H, J=6.6 Hz), 0.38 (s, 18H).

### Step d): 4,8-Bis-(5-bromo-thieno[3,2-b]thiophen-2-yl)-6-(3,7-dimethyl-octyl)-[1,2,5]thiadiazolo[3,4-e]isoindole-5,7-dione

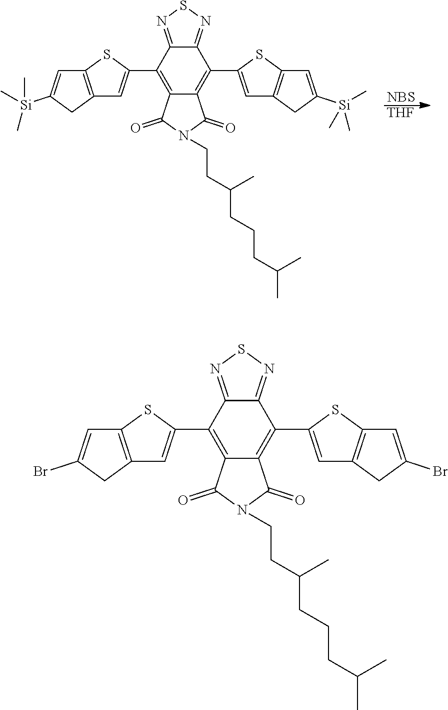

6-(3,7-Dimethyl-octyl)-4,8-bis-(5-trimethylsilanyl-thieno[3,2-b]thiophen-2-yl)-[1,2,5]thiadiazolo[3,4-e]isoindole-5,7-dione (1029 mg; 1.34 mmol) is dissolved in tetrahydrofuran (45.5 cm³). 1-Bromo-pyrrolidine-2,5-dione (490 mg; 2.75 mmol; 2.05 eq.) is added in one portion and the mixture stirred for 18 h. The solvent is removed on a rotary evaporator and the residue is dissolved in dichloromethane (400 cm³), washed with water (200 cm³) and dried over MgSO₄. The solution is filtered and the solvent removed in vacuo. The residue is purified by column chromatography on silica, using petroleum ether (40-60° C.) and dichloromethane as eluents and subsequent recrystallization from boiling cyclohexane. Yield: 551 mg (52.6%).

¹H-NMR (300 MHz, CDCl₃, δ ppm): 8.03 (s, 2H), 7.37 (s, 2H), 3.76 (m, 2H), 1.69 (m, 1H), 1.49 (m, 4H), 1.19 (m, 6H), 0.95 (d, 3H, J=6.2 Hz), 0.84 (d, 6H, J=6.6 Hz).

### EXAMPLE 4

### 4,8-Bis-(5-bromo-4-methyl-thiophen-2-yl)-6-(3,7-dimethyl-octyl)-[1,2,5]thiadiazolo[3,4-e]isoindole-5,7-dione is prepared as follows:

### Step a1): [1,2,5]Thiadiazolo[3,4-e]isoindole-5,7-dione is prepared as in step a1) of Example 1.

### Step a2): 6-(3,7-Dimethyl-octyl)-[1,2,5]thiadiazolo[3,4-e]isoindole-5,7-dione is prepared as in step a2) of Example 3.

### Step c): 6-(3,7-Dimethyl-octyl)-4,8-bis-(4-methyl-5-triisopropylsilanyl-thiophen-2-yl)-[1,2,5]thiadiazolo[3,4-e]isoindole-5,7-dione

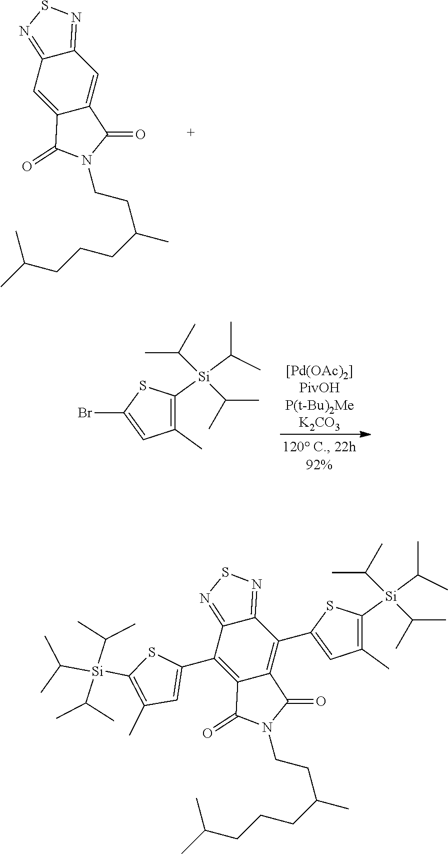 [SMILES: CC(C)CCCC(C)CCN1C(=O)c2cc3nsnc3cc2C1=O ; Cc1cc(Br)sc1[Si](C(C)C)(C(C)C)C(C)C ; Cc1cc(-c2c3c(c(-c4cc(C)c([Si](C(C)C)(C(C)C)C(C)C)s4)c4nsnc24)C(=O)N(CCC(C)CCCC(C)C)C3=O)sc1[Si](C(C)C)(C(C)C)C(C)C]

6-(3,7-Dimethyl-octyl)-[1,2,5]thiadiazolo[3,4-e]isoindole-5,7-dione (4.00 g; 11.58 mmol; 1.00 eq.), 5-bromo-3-methyl-thiophen-2-yl)-triisopropyl-silane (10.38 g; 31.15 mmol; 2.69 eq.), 2,2-dimethyl-propionic acid (1.18 g; 11.58 mmol; 1.00 eq.), palladium(II) acetate (0.26 g; 1.16 mmol; 0.10 eq.), di-tert-butyl-methyl-phosphane tetrafluoroborate (0.57 g; 2.32 mmol; 0.20 eq.) and potassium carbonate (4.80 g; 34.74 mmol; 3.00 eq.) are dissolved in anhydrous toluene (13.55 cm³) and degassed. The reaction mixture is heated to 120° C. for 18 h, under nitrogen. The mixture is cooled to room temperature, diluted with dichloromethane, filtered and the solvent is removed under reduced pressure. The residue is dissolved in petroleum ether (40-60° C.) (10 cm³) and purified via column chromatography on silica, using petroleum ether (40-60° C.) and dichloromethane as eluents. The product is obtained as an orange solid, 9.1 g (92%). ¹H NMR (400 MHz, CDCl₃, δ ppm): 7.86 (s, 2H), 3.81-3.68 (m, 2H), 2.47 (s, 6H), 1.73-1.68 (m, 1H), 1.57-1.47 (m, 10H), 1.34 (m, 2H), 1.19 (d, J=7.5 Hz, 36H), 1.15-1.02 (m, 3H) 0.96 (d, J=6.1 Hz, 3H), 0.85 (d, J=6.6 Hz, 6H)

### Deprotection step): 6-(3,7-Dimethyl-octyl)-4,8-bis-(4-methyl-thiophen-2-yl)-[1,2,5]thiadiazolo[3,4-e]isoindole-5,7-dione

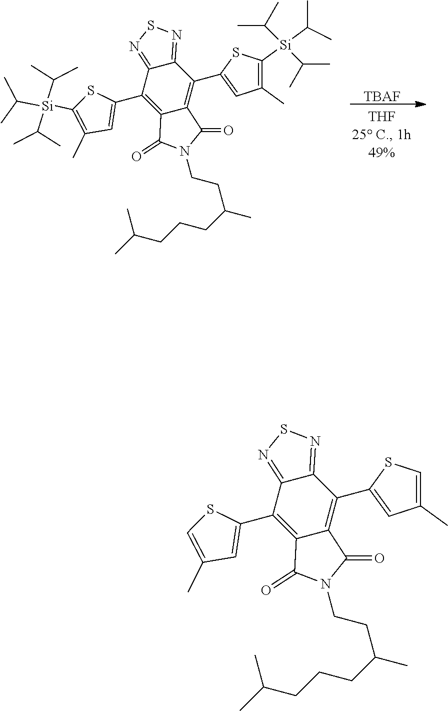

6-(3,7-Dimethyl-octyl)-4,8-bis-(4-methyl-5-triisopropylsilanyl-thiophen-2-yl)-[1,2,5]thiadiazolo[3,4-e]isoindole-5,7-dione (3.09 g; 3.63 mmol; 1.00 eq.) is dissolved in tetrahydrofuran (20 cm³) and tetrabutylammonium fluoride (1 M in tetrahydrofuran, 11.00 cm³; 11.00 mmol; 1.00 eq) is added dropwise. The reaction mixture is allowed to stir for 1 hour at room temperature. Water (50 cm³) is added and the reaction is extracted with chloroform. The organic layer is dried over magnesium sulphate, filtered and concentrated under reduced pressure. The crude product is purified by column chromatography on silica using petroleum ether (40-60° C.): dichloromethane as eluent run on a gradient of 0-30% dichloromethane. The product is obtained as an orange oil, 0.95 g (49%). ¹H NMR (400 MHz, CDCl₃, δ ppm):7.67 (d, J=1.5 Hz, 2H), 7.29 (p, J=1.0 Hz, 2H), 3.74 (m, 2H), 2.41 (d, J=1 Hz, 6H), 1.69 (m, 1H), 1.52-1.42 (m, 2H), 1.35-1.22 (m, 2H), 1.13 (m, 1H), 1.05 (m, 4H), 0.95 (d, J=6.2 Hz, 3H), 0.85 (d, J=6.6 Hz, 6H).

### Step d): 4,8-Bis-(5-bromo-4-methyl-thiophen-2-yl)-6-(3,7-dimethyl-octyl)-[1,2,5]thiadiazolo[3,4-e]isoindole-5,7-dione

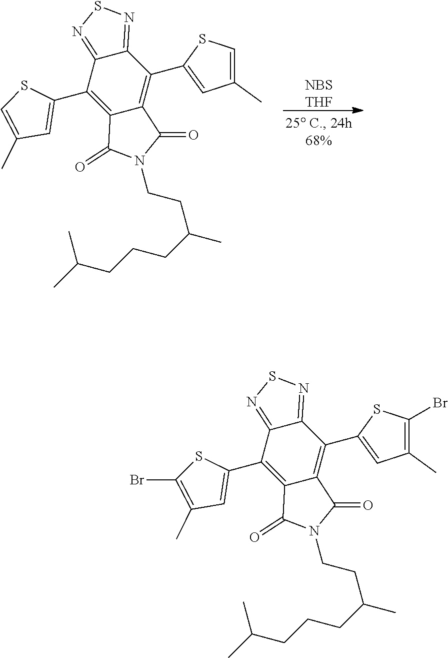

6-(3,7-Dimethyl-octyl)-4,8-bis-(4-methyl-thiophen-2-yl)-[1,2,5]thiadiazolo[3,4-e]isoindole-5,7-dione (0.94 g; 1.75 mmol; 1.00 eq.) is dissolved in tetrahydrofuran (10 cm³) and 1-bromo-pyrrolidine-2,5-dione (0.68 g; 3.85 mmol; 2.20 eq.) is added in one portion. The mixture is stirred in darkness for 24 hours at room temperature. The reaction mixture is evaporated to dryness and the residue is dissolved in dichloromethane (100 cm³), washed with water and dried over magnesium sulphate. The solution is filtered and evaporated to dryness. The crude product is purified by column chromatography on silica using petroleum ether (40-60° C.): dichloromethane as eluent run on a gradient of 0-40% dichloromethane. The product is further purified by recrystallisation using dichloromethane and acetonitrile to give the product as a red solid 0.83 g (68%). ¹H NMR (400 MHz, CDCl₃, δ ppm): 7.69 (s, 2H), 3.75 (m, 2H), 2.33 (s, 6H), 1.68 (m, 1H), 1.56-1.46 (m, 4H), 1.36-1.21 (m, 3H), 1.17-1.09 (m, 2H), 0.96 (d, J=6.1 Hz, 3H), 0.85 (d, J=6.6 Hz, 6H).

1. A process of preparing a compound of formula I 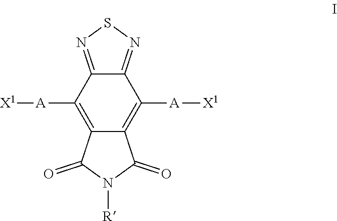
comprising the steps of reacting a compound of formula I1 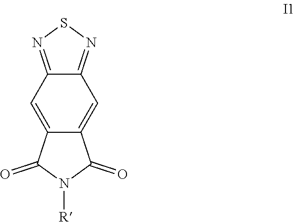
with an aryl- or heteroaryl compound Pg-A-X² to give a compound of formula I2, 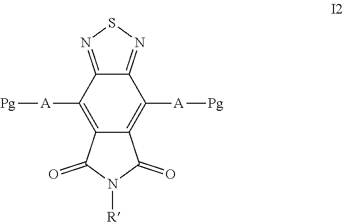
and replacing the groups Pg in the compound of formula I2 by halide groups X¹, wherein the individual radicals, independently of each other, and on each occurrence identically or differently, have the following meanings
A is arylene or heteroarylene with 5 to 30 ring atoms that is optionally substituted, preferably by one or more groups R^{S},
R′ is H or has one of the meanings of R,
R is straight-chain, branched or cyclic alkyl with 1 to 30 C atoms, in which one or more CH₂ groups are optionally replaced by —O—, —S—, —C(═O)—, —C(═S)—, —C(═O)—O—, —O—C(═O)—, —NR⁰—, —SiR⁰R⁰⁰—, —CF₂—, —CHR⁰═CR⁰⁰—, —CY¹═CY²— or —C≡C— in such a manner that O and/or S atoms are not linked directly to one another, and in which one or more H atoms are optionally replaced by F, Cl, Br, I or CN, or denotes aryl or heteroaryl with 5 to 15 ring atoms, which is mono- or polycyclic and unsubstituted or substituted by one or more groups R^{S},
R^{S} is F, Br, Cl, —CN, —NC, —NCO, —NCS, —OCN, —SCN, —C(O)NR⁰R⁰⁰, —C(O)X⁰, —C(O)R⁰, —C(O)OR⁰, —NH₂, —NR⁰R⁰⁰, —SH, —SR⁰, —SO₃H, —SO₂R⁰, —OH, —NO₂, —CF₃, —SF_{S}, optionally substituted silyl, carbyl or hydrocarbyl with 1 to 40 C atoms that is optionally substituted and optionally comprises one or more hetero atoms,
R⁰ and R⁰⁰ are, independently of each other, H or optionally substituted C₁₋₄₀ carbyl or hydrocarbyl, and preferably denote H or alkyl with 1 to 12 C-atoms,
X⁰ is halogen,
X¹ is halogen,
X² is a leaving group,
Pg is H or a protecting group.

2. The process of claim 1, comprising the following steps:
a1) Reacting benzo[2,1,3]thiadiazole 1 that is substituted in 5- and 6-position by Cl, Br, I or sulfonate, preferably by Cl, Br, I, triflate, nonaflate or tosylate, with a cyanating agent, optionally in the presence of a catalyst, to give a product mixture of 5,6-dicyano-benzo [2,1,3] thia-diazole 2 and [1,2,5] thiadiazolo [3,4-e]isoindole-5,7-dione 3, and adding an acid or an acid chloride to the product mixture, and
a2) optionally adding a substituent R to the N-atom in 6-position of the [1,2,5]thiadiazolo[3,4-e]isoindole-5,7-dione 3 to give the N-substituted [1,2,5]thiadiazolo[3,4-e]isoindole-5,7-dione 4,
or, alternatively to steps a1) and a2),
b) reacting benzo[2,1,3]thiadiazole 1 that is substituted in 5- and 6-position by Cl, Br, I or sulfonate with a primary amine R—NH₂ and CO in the presence of a catalyst to give N-substituted [1,2,5]thiadiazolo[3,4-e]isoindole-5,7-dione 4,
and, subsequently to steps a1) and a2) or step b),
c) reacting the [1,2,5]thiadiazolo[3,4-e]isoindole-5,7-dione 3 of step a1), or the N-substituted [1,2,5]thiadiazolo[3,4-e]isoindole-5,7-dione 4 of step a2) or b), with an aryl- or heteroaryl compound Pg-A-X² in the presence of a catalyst and an additive consisting of or comprising a base, to give [1,2,5]thiadiazolo[3,4-e]isoindole-5,7-dione 5 that is 4,8-disubstituted with -A-Pg and optionally N-substituted, and
d) reacting the product 5 from step c) with a halogenating agent containing a halide group X¹, to give 4,8-dihalo-[1,2,5]thia-diazolo[3,4-e]isoindole-5,7-dione 6 that is optionally N-substituted,
wherein the individual radicals are as defined for the compound of Formula I.

3. The process of claim 2, wherein in step d), before reaction with the halogenating agent, the product 5 from step c) is reacted with a deprotecting agent.

4. The process according to claim 1, wherein R′ has one of the meanings of R.

5. The process according to claim 1, wherein R is selected from the group consisting of straight-chain or branched alkyl, alkoxy or sulfanylalkyl with 1 to 30 C atoms, and straight-chain or branched alkylcarbonyl, alkylcarbonyloxy or alkyloxycarbonyl with 2 to 30 C atoms, each of the aforementioned groups being unsubstituted or substituted by one or more F atoms, aryl, heteroaryl, aryloxy and heteroaryloxy, each of which is optionally fluorinated, alkylated or alkoxylated and has 4 to 30 ring atoms.

6. The process according to claim 1, wherein X¹ is Br or I.

7. The process according to claim 1, wherein R^{S} denotes, on each occurrence identically or differently, H, straight-chain, branched or cyclic alkyl with 1 to 30 C atoms, in which one or more CH₂ groups are optionally replaced by —O—, —S—, —C(O)—, —C(S)—, —C(O)—O—, —O—C(O)—, —NR⁰—, —SiR⁰R⁰⁰—, —CF₂—, —CHR⁰═CR⁰⁰—, —CY¹═CY²— or —C≡C— in such a manner that O and/or S atoms are not linked directly to one another, and in which one or more H atoms are optionally replaced by F, Cl, Br, I or CN, or denotes aryl, heteroaryl, aryloxy or heteroaryloxy with 4 to 20 ring atoms which is optionally substituted, preferably by halogen or by one or more of the aforementioned alkyl or cyclic alkyl groups.

8. The process according to claim 1, wherein A is selected from the following formulae 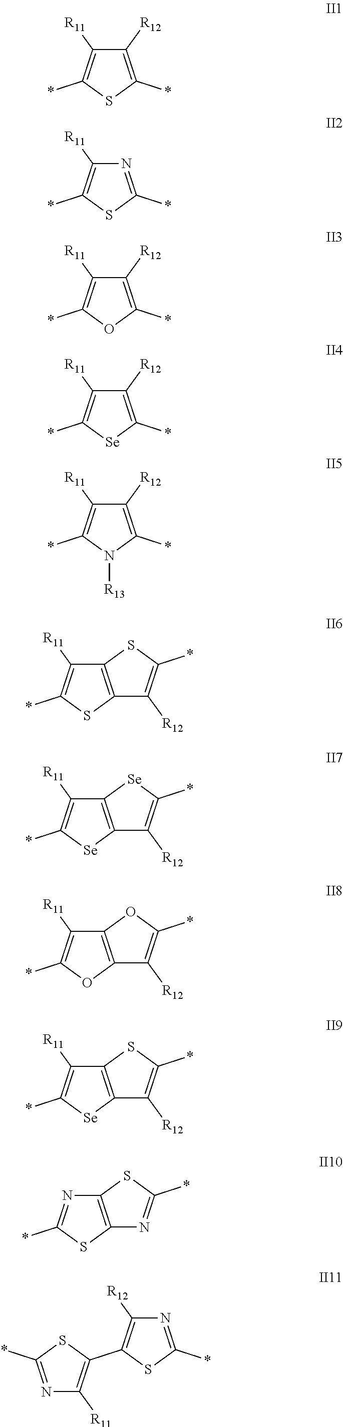 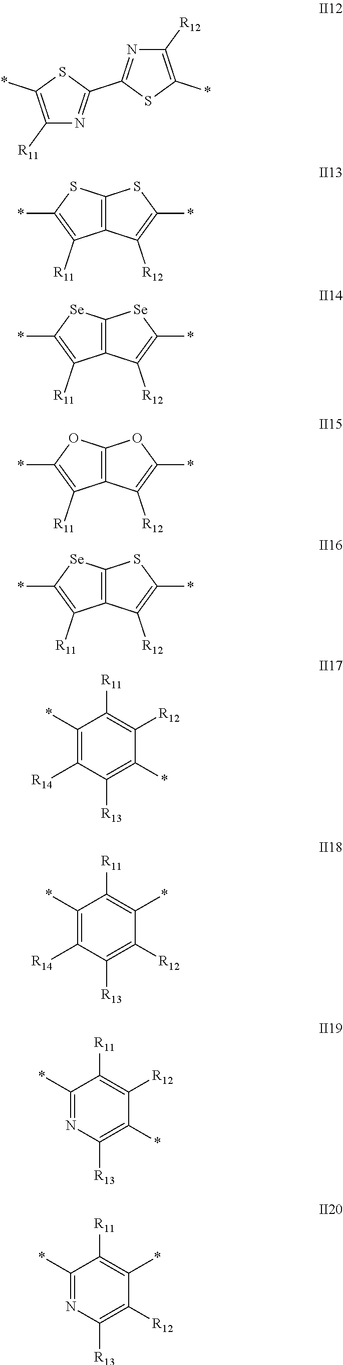 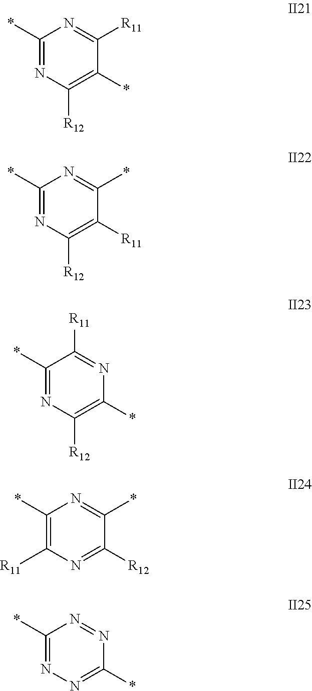
wherein RH, R¹², R¹³ and R¹⁴ independently of each other denote H or have one of the meanings of R^{S} as defined for the respond of Formula I.

9. The process according to claim 1, wherein X² is a leaving group selected from the group consisting of H, Cl, Br, I, O-tosylate, O-triflate, O-mesylate, O-nonaflate, —O—SO₂Z¹, —Si(Z¹)₃, —SiMe₂F, —SiMeF₂, wherein Me denotes a methyl group, and Z¹ is selected from the group consisting of C₁₋₁₀ alkyl and C₆₋₁₂ aryl, each being optionally substituted, and two groups Z¹ may also form a cyclic group.

10. The process according to claim 1, wherein Pg is H or a protecting group selected from the group consisting of an activated C—H bond, Cl, Br, I, O-tosylate, O-triflate, O-mesylate, O-nonaflate, —O—SO₂Z¹, —Si(Z¹)₃, —SiMe₂F, —SiMeF₂, wherein Me denotes a methyl group, and Z¹ is selected from the group consisting of C₁₋₁₀ alkyl and C₆₋₁₂ aryl, each being optionally substituted, and two groups Z¹ may also form a cyclic group.

11. The process according to claim 2, wherein the cyanating agent in step a1) is selected from CuCN, KCN and NaCN.

12. The process according to claim 2, wherein the acid or acid chloride in step a1) is a mineral acid, BF₃, SOCl₂ or oxalyl chloride.

13. The process according to claim 1, wherein the catalyst in one or more of the reaction of the compound of formula I1 with Pg-A-X², step a1), step b) and step c), is a palladium(0) catalyst or palladium(II) catalyst.

14. The process according to claim 13, wherein the catalyst is a palladium(0) catalyst or palladium(II) catalyst comprising a trisubstituted phosphine ligand.

15. The process according to claim 14, wherein the trisubstituted phosphine ligand is selected from the formula R^{a}ₓR^{b}_{y}R^{c}_{z}P, wherein P denotes phosphorus, R^{a}, R^{b} and R^{c} are identical or different straight-chain, branched or cyclic alkyl groups with 1 to 12 C atoms that are optionally fluorinated, or aryl groups with 4 to 20 C atoms that are optionally substituted, and x, y and z are 0, 1, 2 or 3, with x+y+z=3, or selected from the formula Ph₂P(CH₂)ₙPPh₂ where n is an integer from 1 to 5, and any substituted derivatives thereof.

16. The process according to claim 13, wherein the trisubstituted phosphine ligand is formed in situ from the corresponding phosphonium salt in the presence of a base.

17. The process according to claim 1, wherein the reaction of the compound of formula I1 with Pg-A-X², or step c), is carried out in the presence of a base selected from hydroxides, carboxylates, carbonates, fluorides and phosphates of caesium, an alkali metal or an alkaline earth metal.

18. The process according to claim 1, wherein the reaction of the compound of formula I1 with Pg-A-X², or step c), is carried out in the presence of an additive selected from the following groups:
the group consisting of caesium bases,
the group consisting of anions which are generated from pivalic acid, pivalic acid derivatives, or R^{s}—COOH, and an anhydrous base,
the group consisting of additives comprising a silver salt and an anhydrous base or R^{S}₄NOH,
wherein R^{S} is as defined for the compound of formula I.

19. The process according to claim 2, wherein steps a1) and a2) are carried out in a solvent selected from DMF, nitrobenzene, NMP, dimethylacetamide, toluene, xylene (o-, m-, p- or mixtures thereof), mesitylene, isopropylbenzene and dichloromethane.

20. The process according to claim 2, wherein step b) is carried out in a solvent selected from tetrahydrofuran, 2-methyltetrahydrofuran, DMF, nitrobenzene, NMP, dimethylacetamide, toluene, xylene (o-, m-, p- or mixtures thereof), mesitylene, and isopropylbenzene.

21. The process according to claim 2, wherein step c) is carried out in a solvent selected from DMF, nitrobenzene, NMP, dimethylacetamide, toluene, xylene (o-, m-, p- or mixtures thereof), mesitylene, and isopropylbenzene.
